(19) United States
(12) Patent Application Publication
(10) Pub. No.: US 2005/0026811 A1
(43) Pub. Date: Feb. 3, 2005

(54) Rage antagonists as agents to reverse amyloidosis and diseases associated therewith

(76) Inventors: Adnan M. M. Mjalli, Jamestown, NC (US); Robert C. Andrews, Jamestown, NC (US); Jane M. Shen, Winston-Salem, NC (US); Robert Rothlein, Summerfield, NC (US)

Correspondence Address: KILPATRICK STOCKTON LLP, 1001 WEST FOURTH STREET, WINSTON-SALEM, NC, 27101, US

(21) Appl. No.: 10/850,238

(22) Filed: May 20, 2004

Related U.S. Application Data
(60) Provisional application No. 60/471,969, filed on May 20, 2003.

(51) Int. Cl. A61K 31/425; A61K 38/00

(52) U.S. Cl. 514/2

(57) ABSTRACT

Disclosed are RAGE antagonist compounds that have the ability to reverse pre-existing amyloidosis. Treatment with the RAGE antagonist compounds described herein may be used to reduce plaque size and improve cognition for subjects in the later stages of Alzheimer's disease. Additionally, the RAGE antagonists described herein may be used to reduce the onset of plaque formation and thereby prevent loss of cognition and other symptoms associated with Alzheimer's Disease and other diseases of amyloid deposition.

### RELATED APPLICATIONS

This application claims priority to U.S. Provisional Patent Application Ser. No. 60/471,969, filed May 20, 2003. The disclosure of U.S. Provisional Patent Application Ser. No. 60/471,969 is hereby incorporated by reference in its entirety herein.

### FIELD OF THE INVENTION

The present invention relates to therapeutics that reduce amyloid plaques and reverse symptoms associated with amyloidosis. More particularly, the present invention comprises the use of antagonists for the Receptor for Advanced Glycated Endproducts (RAGE) for the reversal of symptoms of β-amyloidosis such as Alzheimer's Disease.

### BACKGROUND OF THE INVENTION

Senile plaques containing amyloid-β (Aβ peptide are one of the neuropathological hallmarks of Alzheimer's disease (AD). Considerable effort has been expended in understanding the relationship of Aβ and Aβ-containing senile plaques to AD. Much of this work has focused on the biosynthesis of Aβ and factors that influence its deposition (Selkoe, Nature 399:A23-31 (1999)). The Aβ peptides are primarily two peptides of either 40 or 42 amino acids generated via internal proteolysis of the amyloid precursor protein (APP) (Checler, Neurochem., 65:1431-1444 (1995); Wang, et al., J. Biol. Chem., 271:31894-31902 (1996)). In addition to Aβ-containing senile plaques, a variety of neuronal cytoskeletal alterations are prominent features of AD neuropathology. These include the presence of phospho-tau containing neurofibrillary tangles, dystrophic neurites (both free-lying and those present in neuritic senile plaques), and synapse loss (Selkoe, Neuron, 6:487-498 (1991); Galasko et al., Arch. Neurol., 51:888-895 (1994)). Whether these abnormal features are the result, or the cause, of neuronal loss is still controversial. Regardless of the precise mechanism, the neuronal and synaptic loss which occurs with development of AD leads to cognitive decline (Selkoe, Ann. Rev. Neurosci., 17:489-517 (1994)).

Early onset autosomal dominant AD is directly linked to mutations in one of several genes: APP, presenilin 1 (PS1), or presenilin 2 (PS2) (St. George-Hyslop, P. H., The Molecular Genetics of Alzheimer's Disease, New York: Raven Press (1993); Sherrington et al., Nature, 375:754-760 (1995); Levy-Lahad et al., Science, 269:973-977 (1995); Rogaev et al., Nature, 376:775-778 (1995)). In addition, several risk factor genes, most notably the APOE4 allele, alter risk for later onset AD (Wisniewski et al., Neurosci. Lett., 135:235-238 (1992); Strittmatter et al., Proc. Natl. Acad. Sci. USA, 90:1977-1981 (1993)), and it is clear that mutations or polymorphisms in several other genes can lead to similar AD phenotypes.

There is some controversy as to whether Aβ causes AD. Still, there are several indications that amyloid deposition plays an important role in AD. First, mutations in the APP gene appear to segregate within families affected with familial AD (Cahrtier-Harline et al., Nature, 353:844-846 (1991); Kennedy et al., Brain, 116:309-324 (1993)). Also, amyloid deposition temporally precedes the development of neurofibrillary changes (Pappolla et al., Mol. Chem. Neuropathol., 28:21-34, (1996)). Finally, Aβ has been shown to be toxic to neurons (Yankner et al., Science, 250:279-282 (1990); Behl et al., Cell, 77:817-827 (1992); Behl et al., Brain Res., 645:253-264 (1994); and Zhang et al., Comp. Biochem. Biophys., 106:165-170 (1994)).

Several groups have described approaches for preventing amyloid plaques from forming, or for reducing pathogenic activation of cellular pathways as a result of amyloid plaque formation. For example, U.S. Pat. Nos. 6,221,667 and 6,472,145 describe the use of mobile ionophores to modulate APP catabolism and subsequent amyloid deposition. U.S. Pat. No. 5,840,294 describes the use of sulfonates and sulfates to inhibit amyloid deposition. U.S. Pat. No. 5,817,626 describes the use of biotinylated Aβ peptides to inhibit Aβ peptide aggregation, and U.S. Pat. No. 6,441,049 describes the use of compounds that inhibit the interaction of Aβ with nicotinic acetylcholine receptors. Also, U.S. Pat. No. 6,323,218 describes the identification of pharmacological agents that inhibit Aβ-mediated production of radical oxygen species. U.S. Pat. No. 6,274,615 describes the use of melatonin to inhibit or reverse the formation of fibrils or amyloid deposits associated with amyloidosis-related disorders.

Still, as yet there are no treatments which are clinically effective in preventing or reversing symptoms, such as cognitive loss, associated with Aβ plaque formation. Although genetic testing for AD may be used for prognostic purposes, it does not provide a cure for the disease. In addition, the onset of AD is not always clear-cut. There may be an extended period of time when an individual may not realize that plaque deposition and associated cognitive loss has ensued. Thus, there is a need for methods and compositions to reduce the extent of amyloid plaque formation and to reduce already formed plaques in patients suffering from AD and other diseases of amyloidosis.

### SUMMARY OF THE INVENTION

The present invention comprises methods and compositions that reverse amyloidosis and conditions and diseases associated therewith. Thus, embodiments of the present invention comprise the use of antagonists for the Receptor for Advanced Glycated Endproducts (RAGE) for the prevention and/or reversal of symptoms of amyloidosis such as Alzheimer's Disease.

For example, in one embodiment, the present invention comprises a composition to reverse pre-existing amyloidosis in an individual in need thereof comprising a pharmacologically effective amount of a RAGE antagonist in a pharmaceutically acceptable carrier, wherein a pharmacologically effective amount of RAGE antagonist comprises sufficient RAGE antagonist to reduce pre-existing amyloid plaques in the individual.

In another embodiment, the present invention comprises a composition to inhibit the onset and/or progression of amyloidosis in an individual comprising a pharmacologically effective amount of a RAGE antagonist in a pharmaceutically acceptable carrier, wherein a pharmacologically effective amount of antagonist comprises sufficient RAGE antagonist to reduce amyloid plaque formation in the individual.

The present invention also describes methods for reducing amyloidosis or preventing the onset of amyloidosis. In another embodiment, the present invention comprises a method to reverse pre-existing amyloidosis in an individual in need thereof comprising administering a pharmacologically effective amount of a RAGE antagonist in a pharmaceutically acceptable carrier to the individual, wherein a pharmacologically effective amount of RAGE antagonist comprises sufficient RAGE antagonist to reduce pre-existing amyloid plaques in the individual.

In yet another embodiment, the present invention comprises a method to inhibit the onset and/or progression of amyloidosis in an individual comprising administering a pharmacologically effective amount of a RAGE antagonist in a pharmaceutically acceptable carrier to the individual, wherein a pharmacologically effective amount of antagonist comprises sufficient RAGE antagonist to reduce amyloid plaque formation in the individual.

Thus, an object of the present invention is to provide methods and compositions for the prevention and reversal of symptoms associated with amyloidosis, such as Alzheimer's disease. There are, of course, additional features of the invention which will be described hereinafter. It is to be understood that the invention is not limited in its application to the specific details as set forth in the following description and figures.

The invention is capable of other embodiments and of being practiced or carried out in various ways.

### BRIEF DESCRIPTION OF THE FIGURES

FIG. 1 illustrates the effect of RAGE antagonist compounds Example A and Example B on amyloid-β (Aβ) deposition in an APP mouse model of later-stage Alzheimer's Disease (AD) in accordance with an embodiment of the present invention. Panel A shows results for each experimental group, and panel B shows results for each individual animal. Administration of either saline vehicle or a RAGE-antagonist in saline began at 12 months of age (12 m) and continued until 15 months of age (15 m). The RAGE antagonist compounds were administered by intraperitoneal injection (i.p.) or orally (p.o.) in doses ranging from 5 mg/kg/day to 20 mg/kg/day as indicated. Animals were sacrificed at 15 months (day 90) and processed by immunohistochemistry to determine the amyloid burden. The 15 m control corresponds to mice injected with 100 μl of saline/mouse/day, and the 12 m control corresponds to 12 month old AAP mice used as the zero timepoint. p<0.001 for all groups compared to vehicle control.

FIG. 2 illustrates the effect of RAGE antagonist compounds Example B, Example C, and Example D, on Aβ deposition in an APP mouse model of early-stage Alzheimer's Disease (AD) in accordance with an embodiment of the present invention. Panel A shows the results for each experimental group, and panel B shows results for each individual animal. Vehicle (saline) or RAGE antagonist compounds were administered by intraperitoneal (i.p.) injection of 5 mg/kg/day of the indicated compound for 90 days, starting at 6 months of age and continued until 9 months of age. Animals were sacrificed on day 90 and processed to determine the amyloid burden. p<0.001 for all compound groups as compared to the vehicle control.

FIG. 3 illustrates the effect of RAGE antagonist compounds Example A and Example B on cognition in mice with established, later-stage AD, measured as the latency time to find a hidden platform in a Morris water maze, in accordance with an embodiment of the present invention. The mice used were the same mice used for determination of amyloid load as described in FIG. 1; cognitive function was measured prior to sacrifice. p<0.001 for all compound groups compared to vehicle control.

FIG. 4 illustrates the effect of RAGE antagonist compounds Example B, Example C, and Example D, on cognition in mice with early-stage AD, measured as latency time to find a hidden platform in a Morris water maze, in accordance with an embodiment of the present invention. Panel A shows the results for each experimental group, and panel B shows the results for each individual animal. The mice used were the same mice used for determination of amyloid load as described in FIG. 2; cognitive function was measured prior to sacrifice. p<0.001 for compound groups compared to vehicle control.

### DETAILED DESCRIPTION OF THE INVENTION

The present invention comprises methods and compositions to reverse amyloid plaque deposition and to reverse symptoms associated with amyloidosis. Also, the present invention comprises methods and compositions to inhibit amyloid plaque deposition and symptoms associated with excess amyloid plaque formation. For example, the methods and compositions of the present invention inhibit amyloid-β (Aβ plaque formation, reduce the size of pre-existing Aβ plaques, and reverse symptoms associated with Alzheimer's Disease.

In one embodiment, the present invention comprises a composition to reverse pre-existing amyloidosis in an individual in need thereof comprising a pharmacologically effective amount of a RAGE antagonist in a pharmaceutically acceptable carrier, wherein a pharmacologically effective amount of antagonist comprises sufficient RAGE antagonist to reduce pre-existing amyloid plaques in the individual. In an embodiment, a pharmacologically effective amount of the RAGE antagonist reverses symptoms associated with amyloidosis.

In an embodiment, the individual is suffering from a disease of abnormal amyloid accumulation. For example, the amyloid plaque may comprises an amyloid-β (Aβ plaque. In an embodiment, the plaque reduction occurs, at least in part, in the individual's brain. For example, the amyloidosis may cause Alzheimer's Disease (AD). Thus, in an embodiment, reversal of symptoms associated with amyloidosis is associated with improved cognition.

Alternatively and/or additionally, the amyloidosis may be associated with systemic amyloid deposition. Thus, in an embodiment, the amyloidosis comprises amyloid-light chain amyloidosis (AL amyloidosis) or amyloid-associated amyloidosis (AA amyloidosis).

Thus, the present invention may comprise the use of antagonists of the RAGE receptor to reduce pre-existing amyloid plaques in an individual suffering from amyloidosis. In an embodiment, the antagonists bind with high specificity to RAGE. The RAGE antagonists used to reverse amyloidosis and reduce the size of pre-existing plaques may comprise a variety of chemical structures. In an embodiment, the RAGE antagonist may comprise an organic compound having a molecular weight less than 1000 Da. For example, the RAGE antagonist may comprise compounds of Formulas (I), (II), (III) or (IV), such as Example A, Example B, Example C, or Example D, described herein.

Alternatively, the RAGE antagonist may comprise a polypeptide or peptidomimetic. It has been found that certain RAGE fragments act to antagonize the biological function of the receptor by competing for AGEs and other RAGE ligands. Thus, in an embodiment, the RAGE antagonist for reversing plaque formation comprises naturally occurring soluble receptor for advanced glycation endproduct (sRAGE) or a fragment thereof (Neeper et al., 1992). In another embodiment, the RAGE antagonist for reversing plaque formation comprises the 120 amino acid V-domain of RAGE (Neeper et al., (1992) or a fragment thereof. In an embodiment, the sRAGE or a fragment thereof may be linked to an immunoglobulin or immunoglobulin fragment. As used herein, a “fragment” of sRAGE or the V-domain is at least 5 amino acids in length, preferably more than 15 amino acids in length, but is less than the full length polypeptide. Thus, the RAGE antagonist may comprise sRAGE, the V-domain of RAGE, a fragment of sRAGE or the V-domain, or a functional equivalent thereof comprising conservative substitutions, where conservative substitutions are those amino acid substitutions that do not alter biological activity of the peptide. In another embodiment, the RAGE antagonist comprises an anti-RAGE antibody, or a fragment thereof.

The present invention contemplates the use of dosages of RAGE antagonists that are individualized as required by the subject. Thus, in an embodiment, a pharmacologically effective amount of a RAGE antagonist comprises a dose ranging from 0.01 to 500 mg/kg per day. In other embodiments, a pharmacologically effective amount comprises a dose of RAGE antagonist ranging from 0.1 to 200 mg/kg per day. In alternative embodiments, a pharmacologically effective amount may comprise a dose ranging from 1 to 100 mg/kg per day, or from about 5 to about 20 mg/kg per day.

A variety of methods are available to administer the plaque-reversing compositions and compounds of the present invention. In an embodiment, the composition comprising a RAGE antagonist is administered topically. In an embodiment, the composition comprising a RAGE antagonist is administered by an intraperitoneal route. In another embodiment, the RAGE antagonist is administered intravenously. Alternatively, the RAGE antagonist may be administered orally. In other embodiments, the RAGE antagonist is administered subcutaneously or by a transdermal route.

The diseases treated by the compounds of the present invention may respond well to a multi-faceted treatment. Thus, in an embodiment, the composition for reducing pre-existing amyloid plaques comprises a second therapeutic agent. The second therapeutic agent may comprise a compound effective in treating Aβ amyloidosis. For example, the second therapeutic agent may comprise a cholinesterase inhibitor, an antipsychotic, an antidepressant, or an anticonvulsant.

Alternatively and/or additionally, the second therapeutic agent may comprise a compound effective in treating amyloid-light chain (AL) amyloidosis. In example embodiments, the second therapeutic agent may comprise an alkylating agent, an antibiotic, an antimetabolite, a plant alkaloid, a hormone, or a biologic response modifier such as an interferon or an interleukin.

The second therapeutic agent may also comprise a compound effective in treating amyloid-associated (AA) amyloidosis. Thus, the second therapeutic agent may also comprise an analgesic, a nonsteroidal anti-inflammatory drug (NSAID), a disease-modifying antirheumatic drug (DMARD), or a biologic response modifier.

In another embodiment, the present invention comprises a composition to inhibit the onset and/or progression of amyloidosis in an individual comprising a pharmacologically effective amount of a RAGE antagonist in a pharmaceutically acceptable carrier, wherein a pharmacologically effective amount of antagonist comprises sufficient RAGE antagonist to reduce amyloid plaque formation in the individual. In an embodiment, a pharmacologically effective amount of the RAGE antagonist inhibits symptoms associated with amyloidosis.

The individual may be suffering from, or at risk of developing, a disease of abnormal amyloid accumulation. For example, in an embodiment, the amyloid plaque may comprise an amyloid-β (Aβ) plaque. In an embodiment, inhibition of plaque formation may occur, at least in part, in the individual's brain. For example, in an embodiment, the amyloidosis may cause Alzheimer's Disease (AD) and inhibition of symptoms associated with amyloidosis is associated with improved cognition.

Alternatively and/or additionally, the amyloidosis may be associated with systemic amyloid deposition. Thus, in an embodiment, the amyloidosis comprises amyloid-light chain amyloidosis (AL amyloidosis) or amyloid-associated amyloidosis (AA amyloidosis).

Thus, the present invention provides antagonists of the RAGE receptor as agents to inhibit the onset, and/or progression, of amyloid plaque formation. The RAGE antagonist may comprise a low molecular weight (e.g., <1000 molecular weight) organic compound. For example, in an embodiment, the RAGE antagonist comprises compounds of Formulas (I), (II), (III) or (IV), such as Example A, Example B, Example C, or Example D, described herein. Alternatively, the RAGE antagonist may comprise a peptidomimetic.

Dosages of RAGE antagonists used to inhibit amyloid plaque formation may be individualized as required by the subject. Thus, in an embodiment, a pharmacologically effective amount comprises a dose of RAGE antagonist ranging from 0.01 to 500 mg/kg per day. In alternative embodiments, a pharmacologically effective amount may comprise a dose of RAGE antagonist ranging from 0.1 to 200 mg/kg per day, or from 1 to 100 mg/kg per day, or from about 5 to about 20 mg/kg per day.

A variety of methods are available to administer the RAGE antagonists of the present invention for inhibition of plaque formation. In an embodiment, a pharmacologically effective amount of the RAGE antagonist is administered by a topical route. In other embodiments, a pharmacologically effective amount of the RAGE antagonist is administered by an intraperitoneal route or intravenously. Alternatively, the RAGE antagonist may be administered orally. In other embodiments, the RAGE antagonist is administered subcutaneously or by a transdermal route.

The diseases treated by the compounds of the present invention may respond well to a multi-faceted treatment. Thus, in an embodiment, the composition for inhibition of amyloid plaque formation may comprise a second therapeutic agent.

In an embodiment, the second therapeutic agent may comprise a compound effective in treating Aβ amyloidosis. Thus, the second therapeutic agent may comprise a cholinesterase inhibitor, an antipsychotic, an antidepressant, or an anticonvulsant.

Alternatively and/or additionally, the second therapeutic agent may comprise a compound effective in treating amyloid-light chain (AL) amyloidosis. Thus, in this embodiment, the second therapeutic agent may comprise an alkylating agent, an antibiotic, an antimetabolite, a plant alkaloid, a hormone, or a biologic response modifier such as an interferon or an interleukin.

In yet another embodiment, the second therapeutic agent may comprise a compound effective in treating amyloid-associated (AA) amyloidosis. For example, the second therapeutic agent may comprise an analgesic, a nonsteroidal anti-inflammatory drug (NSAID), a disease-modifying antirheumatic drug (DMARD), or a biologic response modifier.

The present invention also comprises methods to prevent or reverse symptoms associated with amyloidosis in an individual. Thus, in another embodiment, the present invention comprises a method to reverse amyloidosis in an individual in need thereof comprising administering a pharmacologically effective amount of a RAGE antagonist in a pharmaceutically acceptable carrier to the individual, wherein a pharmacologically effective amount of RAGE antagonist reduces pre-existing amyloid plaques in the individual. In an embodiment, a pharmacologically effective amount the RAGE antagonist reverses symptoms associated with amyloidosis.

The individual may be suffering from a disease of abnormal amyloid accumulation. In an embodiment, the amyloid plaque may comprises an amyloid-β (Aβ plaque. In an embodiment, the plaque reduction may occur, at least in part, in the individual's brain. For example, in an embodiment, the amyloidosis causes Alzheimer's Disease (AD) and reversal of symptoms associated with amyloidosis is associated with improved cognition.

Alternatively and/or additionally, the amyloidosis may be associated with systemic amyloid deposition. Thus, in an embodiment, the amyloidosis comprises amyloid-light chain amyloidosis (AL amyloidosis) or amyloid-associated amyloidosis (AA amyloidosis).

The RAGE antagonists used to reverse amyloidosis may comprise a variety of chemical structures. In an embodiment, the RAGE antagonist comprises a small (i.e., <1000 molecular weight) organic compound. For example, in an embodiment, the RAGE antagonist comprises compounds of Formulas (I), (II), (III) or (IV), such as Example A, Example B, Example C, or Example D, described herein.

Alternatively, the RAGE antagonist may comprise a polypeptide or peptidomimetic. In yet another embodiment, the RAGE antagonist to reverse amyloidosis comprises sRAGE, the V-domain of RAGE, a fragment of sRAGE or the V-domain, or a functional equivalent thereof comprising conservative substitutions. In an embodiment, the sRAGE or fragment thereof is linked to an immunoglobulin fragement. In another embodiment, the RAGE antagonist comprises an anti-RAGE antibody, or a fragment thereof.

In an embodiment, the doses of RAGE antagonist are individualized as required by the subject. Thus, in an embodiment, a pharmacologically effective amount comprises a dose of RAGE antagonist ranging from 0.01 to 500 mg/kg per day. In other embodiments, a pharmacologically effective amount may comprise a dose of RAGE antagonist ranging from 0.1 to 200 mg/kg per day, or from 1 to 100 mg/kg per day, or from about 5 to about 20 mg/kg per day.

A variety of methods are available to administer the plaque-reversing compositions and compounds of the present invention. In an embodiment, the composition comprising a pharmacologically effective of a RAGE antagonist is administered topically. In an embodiment, the composition comprising a pharmacologically effective of a RAGE antagonist is administered by an intraperitoneal route or intravenously. Alternatively, the RAGE antagonist is administered orally. In other embodiments, the RAGE antagonist is administered subcutaneously or by a transdermal route.

The diseases treated by the methods of the present invention may respond well to a multi-faceted treatment. Thus, the composition of the present invention for reversing pre-existing amyloid plaques may comprise a second therapeutic agent.

In an embodiment, the second therapeutic agent comprises a compound effective in treating Aβ amyloidosis. Thus, the second therapeutic agent may comprise a cholinesterase inhibitor, an antipsychotic, an antidepressant, or an anticonvulsant.

Alternatively and/or additionally, the second therapeutic agent may comprise a compound effective in treating amyloid-light chain (AL) amyloidosis. Thus, the second therapeutic agent may comprise an alkylating agent, an antibiotic, an antimetabolite, a plant alkaloid, a hormone, or a biologic response modifier such as an interferon or an interleukin.

In another embodiment, the second therapeutic agent may comprise a compound effective in treating amyloid-associated (AA) amyloidosis. Thus, the second therapeutic agent may comprise an analgesic, a nonsteroidal anti-inflammatory drug (NSAID), a disease-modifying antirheumatic drug (DMARD), or a biologic response modifier.

In another embodiment, the present invention comprises a method to inhibit the onset and/or progression of amyloidosis in an individual comprising administering a pharmacologically effective amount of a RAGE antagonist in a pharmaceutically acceptable carrier to the individual, wherein a pharmacologically effective amount of RAGE antagonist comprises sufficient RAGE antagonist to reduce amyloid plaque formation in the individual. In an embodiment, a pharmacologically effective amount of antagonist inhibits symptoms associated with amyloidosis.

The individual treated by the methods of the present invention may be suffering from, or at risk of developing, a disease of abnormal amyloid accumulation. In an embodiment, the amyloid plaque comprises an amyloid-β (Aβ plaque. In one embodiment, plaque reduction occurs, at least in part, in the individual's brain. For example, in an embodiment, the amyloidosis causes Alzheimer's Disease (AD) and inhibition of symptoms associated with amyloidosis is associated with improved cognition.

Alternatively and/or additionally, the amyloidosis may be associated with systemic amyloid deposition. Thus, in an embodiment, the amyloidosis comprises amyloid-light chain anyloidosis (AL amyloidosis) or amyloid-associated amyloidosis (AA amyloidosis).

The RAGE antagonists used to inhibit plaque formation may comprise a variety-of chemical structures. In an embodiment, the RAGE antagonist to inhibit plaque formation comprises a small (i.e., <1000 molecular weight) organic compound. For example, in an embodiment, the RAGE antagonist comprises compounds of Formulas (I), (II), (III) or (IV), such as Example A, Example B, Example C, or Example D, described herein. Alternatively, the RAGE antagonist may comprise a polypeptide or peptidomimetic.

The dosages of the RAGE antagonist usde to inhibit plaque formation may be individualized as required by the subject. Thus, a pharmacologically effective amount of RAGE antagonist may comprise a dose ranging from 0.01 to 500 mg/kg per day. In other embodiments, a pharmacologically effective amount comprises a dose of RAGE antagonist ranging from 0.1 to 200 mg/kg per day, or from 1 to 100 mg/kg per day, or from about 5 to about 20 mg/kg per day.

A variety of methods are available to administer the compositions and compounds for inhibition of amyloidosis and plaque formation. In an embodiment, the composition comprising a pharmacologically effective of a RAGE antagonist is administered topically. In another embodiment, the RAGE antagonist is administered intravenously, or by an intraperitoneal route. Alternatively, the RAGE antagonist is administered orally. In other embodiments, the RAGE antagonist is administered subcutaneously or by a transdermal route.

The diseases treated by the methods of the present invention may respond well to a multi-faceted treatment. Thus, the composition of the present invention for inhibition of plaque formation and amyloidosis may comprise a second therapeutic agent.

In an embodiment, the second therapeutic agent may comprise a compound effective in treating Aβ amyloidosis. For example, the second therapeutic agent may comprise a cholinesterase inhibitor, an antipsychotic, an antidepressant, or an anticonvulsant.

Alternatively, and/or additionally, the second therapeutic agent comprises a compound effective in treating amyloid-light chain (AL) amyloidosis. In this embodiment, the second therapeutic agent may comprise an alkylating agent, an antibiotic, an antimetabolite, a plant alkaloid, a hormone, or a biologic response modifier such as an interferon or an interleukin.

In another embodiment, the second therapeutic agent may comprise a compound effective in treating amyloid-associated (AA) amyloidosis. Thus, the second therapeutic agent may comprises an analgesic, a nonsteroidal anti-inflammatory drug (NSAID), a disease-modifying antirheumatic drug (DMARD), or a biologic response modifier.

As described above, embodiments of the present invention comprise the use of small organic RAGE antagonists to inhibit the formation of Aβ plaques and to reduce the size of pre-existing Aβ plaques. Small organic RAGE antagonists may comprise organic compounds of less than 1,000 Dalton (Da) molecular weight. Small organic compounds may include RAGE antagonists such as those described in U.S. patent application Ser. No. 09/799,317, filed Mar. 5, 2001 (U.S. Patent Application Publication No. US 2002/0006957); U.S. patent application Ser. No. 10/091,609, filed Mar. 5, 2002 (U.S. Patent Application Publication No. US 2003/0032663); U.S. patent application Ser. No. 10/091,759, filed Mar. 5, 2002 (U.S. Patent Application Publication No. US 2002/0193432); and U.S. patent application Ser. No. 10/382,203, filed Mar. 5, 2003 (U.S. Patent Application Publication No. US 2004/0082542); each of which are incorporated by reference herein in their entirety.

For example, the small molecule RAGE antagonists, Example A, Example B, Example C, and Example D, described herein, inhibit amyloid plaque formation, reduce the size of pre-existing plaques, and reduce the behavioral effects seen with advanced amyloid deposition. Compounds Example A, Example B, Example C, and Example D described herein have been shown to prevent binding of known ligands to RAGE. Thus, in an assay measuring specific binding of the ligand S-100b to RAGE, Example A inhibits binding with an IC₅₀ of about 1 μM, and Example B, Example C, and Example D inhibit binding of ligands to RAGE with an IC₅₀ of less than 1 μM. IC₅₀ is the concentration of an agent which provides 50% of the total inhibition detected for a biological effect of interest, as for example, 50% inhibition of a known ligand binding to RAGE.

Thus, in an embodiment, the present invention provides azole compounds of Formula (I) for inhibiting amyloid plaque formation and/or reducing the size of pre-existing plaques: 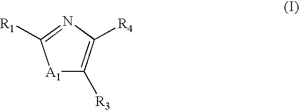
wherein for the compounds of Formula (I):
R₁ comprises -hydrogen, -aryl, -heteroaryl, -cycloalkyl, -heterocyclyl, -alkyl, -alkenyl, -alkynyl, -alkylene-aryl, -alkylene-heteroaryl, -alkylene-heterocyclyl, -alkylene-cycloalkyl, -fused cycloalkylaryl, -fused cycloalkylheteroaryl, -fused heterocyclylaryl, -fused heterocyclylheteroaryl, -alkylene-fused cycloalkylaryl, -alkylene-fused cycloalkylheteroaryl, -alkylene-fused heterocyclylaryl, -alkylene-fused heterocyclylheteroaryl, or -G₁-G₂-G₃—R₅
wherein
   G₁ and G₃ independently comprise alkylene, alkenylene, alkynylene, cycloalkylene, heterocyclylene, arylene, heteroarylene, (aryl)alkylene, (heteroaryl) alkylene, (aryl)alkenylene, (heteroaryl)alkenylene, or a direct bond;
   G₂ comprises —O—, —S—, —S(O)—, —N(R₆)—, —S(O)₂—, —C(O)—, —O—C(O)—, —C(O)—O—, —C(O)N(R₆)—, —N(R₆)C(O)—, —S(O₂)N(R₆)—, N(R₆)S(O₂)—, —O-alkylene-C(O)—, —(O)C-alkylene-O—, —O-alkylene-, -alkylene-O—, alkylene, alkenylene, alkynylene, cycloalkylene, heterocyclylene, arylene, heteroarylene, fused cycloalkylarylene, fused cycloalkylheteroarylene, fused heterocyclylarylene, fused heterocyclylheteroarylene, or a direct bond, wherein R₆ comprises hydrogen, aryl, alkyl, -alkylene-aryl, alkoxy, or -alkylene-O-aryl; and
   R₅ comprises hydrogen, aryl, heteroaryl, cycloalkyl, heterocyclyl, alkyl, alkenyl, alkynyl, -alkylene-aryl, -alkylene-heteroaryl, -alkylene-heterocyclyl, -alkylene-cycloalkyl, fused cycloalkylaryl, fused cycloalkylheteroaryl, fused heterocyclylaryl, fused heterocyclylheteroaryl, -alkylene-fused cycloalkylaryl, -alkylene-fused cycloalkylheteroaryl, -alkylene-fused heterocyclylaryl, or -alkylene-fused heterocyclylheteroaryl;
A₁ comprises O, S, or —N(R₂)—;
wherein
   R₂ comprises
      a) —H;
      b) -aryl;
      c) -heteroaryl;
      d) -cycloalkyl
      e) heterocyclyl;
      f) -alkyl;
      g) -alkenyl;
      h) -alkynyl;
      i) -alkylene-aryl,
      j) -alkylene-heteroaryl,
      k) -alkylene-heterocyclyl,
      l) -alkylene-cycloalkyl;
      m) -fused cycloalkylaryl,
      n) -fused cycloalkylheteroaryl,
      o) -fused heterocyclylaryl,
      p) -fused heterocyclylheteroaryl;
      q) -alkylene-fused cycloalkylaryl,
      r) -alkylene-fused cycloalkylheteroaryl,
      s) -alkylene-fused heterocyclylaryl,
      t) -alkylene-fused heterocyclylheteroaryl; or
      u) a group of the formula 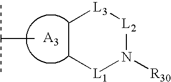
      wherein
         A₃ comprises an aryl or heteroaryl group;
         L₁ and L₂ independently comprise alkylene or alkenylene; and
         L₃ comprises a direct bond, alkylene, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 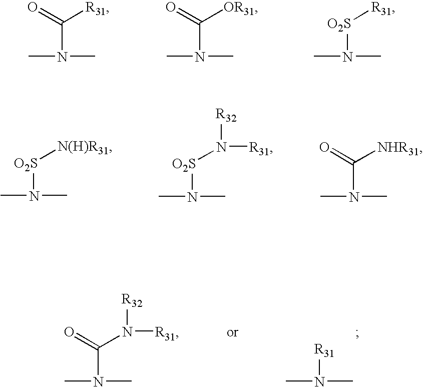
            wherein R₃₀, R₃₁, and R₃₂ independently comprise hydrogen, aryl, heteroaryl, alkyl, alkylene-aryl, or -alkylene-heteroaryl;
R₃ and R₄ independently comprise
   a) -hydrogen,
   b) -halogen,
   c) -hydroxyl,
   d) -cyano,
   e) -carbamoyl,
   f) -carboxyl,
   g) -aryl,
   h) -heteroaryl,
   i) -cycloalkyl,
   j) -heterocyclyl,
   k) -alkyl,
   l) -alkenyl,
   m) -alkynyl,
   n) -alkylene-aryl,
   o) -alkylene-heteroaryl,
   p) -alkylene-heterocyclyl,
   q) -alkylene-cycloalkyl,
   r) -fused cycloalkylaryl,
   s) -fused cycloalkylheteroaryl,
   t) -fused heterocyclylaryl,
   u) -fused heterocyclylheteroaryl,
   v) -alkylene-fused cycloalkylaryl,
   w) -alkylene-fused cycloalkylheteroaryl,
   x) -alkylene-fused heterocyclylaryl,
   y) -alkylene-fused heterocyclylheteroaryl;
   z) —C(O)—O-alkyl;
   aa) —C(O)—O-alkylene-aryl;
   bb) —C(O)—NH-alkyl;
   cc) —C(O)—NH-alkylene-aryl;
   dd) —SO₂-alkyl;
   ee) —SO₂-alkylene-aryl;
   ff) —SO₂-aryl;
   gg) —SO₂—NH-alkyl;
   hh) —SO₂—NH-alkylene-aryl;
   ii) —C(O)-alkyl;
   jj) —C(O)-alkylene-aryl;
   kk) -G₄-G₅-G₆-R₇;
   ll) —Y₁-alkyl;
   mm) —Y₁-aryl;
   nn) —Y₁-heteroaryl;
   oo) —Y₁-alkylene-aryl;
   pp) —Y₁-alkylene-heteroaryl;
   qq) —Y₁-alkylene-NR₉R₁₀; or
   rr) —Y₁-alkylene-W₁—R₁₁;
   wherein
      G₄ and G₆ independently comprise alkylene, alkenylene, alkynylene, cycloalkylene, heterocyclylene, arylene, heteroarylene, (aryl)alkylene, (heteroaryl)alkylene, (aryl)alkenylene, (heteroaryl)alkenylene, or a direct bond;
      G₅ comprises —O—, —S—, —N(R₈)—, —S(O)—, —S(O)₂—, —C(O)—, —O—C(O)—, —C(O)—O—, —C(O)N(R₈)—, N(R₈)C(O)—, —S(O₂)N(R₉)—, N(R₈)S(O₂)—, —O-alkylene-C(O), —(O)C-alkylene-O—, —O-alkylene-, -alkylene-O—, alkylene, alkenylene, alkynylene, cycloalkylene, heterocyclylene, arylene, heteroarylene, fused cycloalkylarylene, fused cycloalkylheteroarylene, fused heterocyclylarylene, fused heterocyclylheteroarylene, or a direct bond, wherein R₈ comprises -hydrogen, -aryl, -alkyl, -alkylene-aryl, or -alkylene-O-aryl;
      R₇ comprises hydrogen, aryl, heteroaryl, cycloalkyl, heterocyclyl, alkyl, alkenyl, alkynyl, alkylene-aryl, -alkylene-heteroaryl, -alkylene-heterocyclyl, -alkylene-cycloalkyl, fused cycloalkylaryl, fused cycloalkylheteroaryl, fused heterocyclylaryl, fused heterocyclylheteroaryl, alkylene-fused cycloalkylaryl, -alkylene-fused cycloalkylheteroaryl, -alkylene-fused heterocyclylaryl, or -alkylene-fused heterocyclylheteroaryl;
      Y₁ and W₁ independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—, 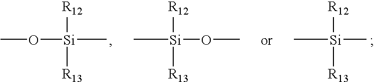
         wherein R₁₂ and R₁₃ independently comprise aryl, alkyl, -alkylene-aryl, alkoxy, or -alkylene-O-aryl; and
      R₉, R₁₀, and R₁₁ independently comprise aryl, heteroaryl, alkyl, -alkylene-heteroaryl, or -alkylene-aryl; and R₉ and R₁₀ may be taken together to form a ring having the formula —(CH₂)ₒ—X₁—(CH₂)ₚ— bonded to the nitrogen atom to which R₉ and R₁₀ are attached,
      wherein
         o and p are, independently, 1, 2, 3, or 4; and
         X₁ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 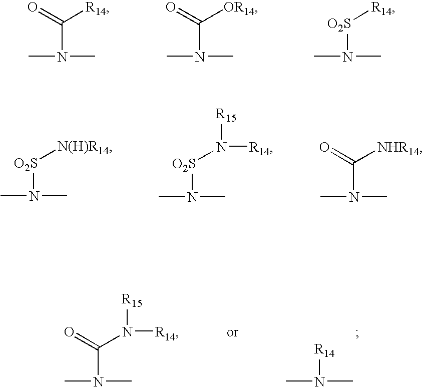
      wherein R₁₄ and R₁₅ independently hydrogen, aryl, heteroaryl, alkyl, -alkylene-aryl, or -alkylene-heteroaryl;
         wherein
the aryl and/or alkyl group(s) in R₁, R₂, R₃, R₄, R₅, R₆, R₇, R₈, R₉, R₁₀, R₁₁, R₁₂, R₁₃, R₁₄, and R₁₅ may be optionally substituted 1-4 times with a substituent group, wherein said substituent group(s) or the term substituted refers to a group comprising:
   a) —H,
   b) -halogen,
   c) -hydroxyl,
   d) -cyano,
   e) -carbamoyl,
   f) -carboxyl,
   g) —Y₂-alkyl;
   h) —Y₂-aryl;
   i) —Y₂-heteroaryl;
   j) —Y₂-alkylene-heteroarylaryl;
   k) —Y₂-alkylene-aryl;
   l) —Y₂-alkylene-W₂—R₁₈;
   m) —Y₃—Y₄—NR₂₃R₂₄,
   n) —Y₃—Y₄—NH—C(═NR₂₅)NR₂₃R₂₄,
   o) —Y₃—Y₄—C(═NR₂₅)NR₂₃R₂₄, or
   p) —Y₃—Y₄—Y₅-A₂,
   wherein
      Y₂ and W₂ independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—, 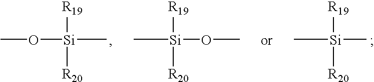
      wherein;
         R₁₉ and R₂₀ independently comprise hydrogen, aryl, alkyl, -alkylene-aryl, alkoxy, or -alkylene-O-aryl; and
      R₁₈ comprises aryl, alkyl, -alkylene-aryl, -alkylene-heteroaryl, and -alkylene-O-aryl;
         Y₃ and Y₅ independently comprise a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—, 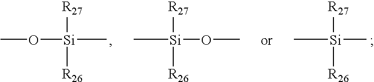
         wherein R₂₇ and R₂₆ independently comprise aryl, alkyl, -alkylene-aryl, alkoxy, or -alkyl-O-aryl;
      Y₄ comprises
         a) -alkylene;
         b) -alkenylene;
         c) -alkynylene;
         d) -arylene;
         e) -heteroarylene;
         f) -cycloalkylene;
         g) -heterocyclylene;
         h) -alkylene-arylene;
         i) -alkylene-heteroarylene;
         j) -alkylene-cycloalkylene;
         k) -alkylene-heterocyclylene;
         l)-arylene-alkylene;
         m) -heteroarylene-alkylene;
         n) -cycloalkylene-alkylene;
         o) -heterocyclylene-alkylene;
         p) —O—;
         q) —S—;
         r) —S(O₂)—; or
         s) —S(O)—;
         wherein said alkylene groups may optionally contain one or more O, S, S(O), or SO₂ atoms;
      A₂ comprises
         a) heterocyclyl, fused arylheterocyclyl, or fused heteroarylheterocyclyl, containing at least one basic nitrogen atom,
         b) -imidazolyl, or
         c) -pyridyl; and
      R₂₃, R₂₄, and R₂₅ independently comprise hydrogen, aryl, heteroaryl, -alkylene-heteroaryl, alkyl, -alkylene-aryl, -alkylene-O-aryl, or -alkylene-O-heteroaryl; and R₂₃ and R₂₄ may be taken together to form a ring having the formula —(CH₂)ₙ—X₃—(CH₂)ₜ— bonded to the nitrogen atom to which R₂₃ and R₂₄ are attached
      wherein
         s and t are, independently, 1, 2, 3, or 4;
         X₃ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 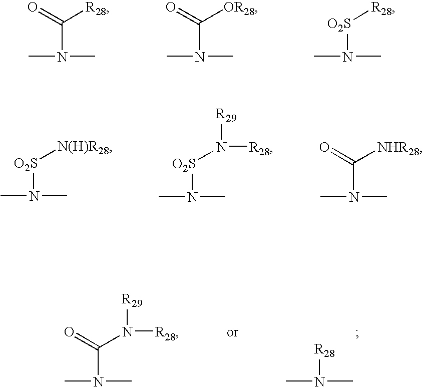
         wherein R₂₈ and R₂₉ independently comprise hydrogen, aryl, heteroaryl, alkyl, -alkylene-aryl, or -alkylene-heteroaryl;
            wherein
            either
   at least one of the groups R₁, R₂, R₃ and R₄ are substituted with at least one group of the formula —Y₃—Y₄—NR₂₃R₂₄, —Y₃—Y₄—NH—C(═NR₂₅)NR₂₃R₂₄, —Y₃—Y₄—C(═NR₂₅)NR₂₃R₂₄, or —Y₃—Y₄—Y₅-A₂, with the proviso that no more than one of R₂₃, R₂₄, and R₂₅ may comprise aryl or heteroaryl; or
   R₂ is a group of the formula 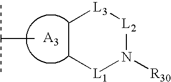
   and
      wherein
   one of R₃ and R₄, R₃ and R₂, or R₁ and R₂ may be taken together to constitute, together with the atoms to which they are bonded, an aryl, heteroaryl, fused arylcycloalkyl, fused arylheterocyclyl, fused heteroarylcycloalkyl, or fused heteroarylheterocyclyl ring system,
   wherein
      said ring system or R₁, R₂, R₃, or R₄ is substituted with at least one group of the formula
         a) —Y₅—Y₆—NR₃₃R₃₄;
         b) —Y₅—Y₆—NH—C(═NR₃₅)NR₃₃R₃₄;
         c) —Y₅—Y₆—C(═NR₃₅)NR₃₃R₃₄; or
         d) —Y₅—Y₆—Y₇-A₄;
      wherein
         Y₅ and Y₇ independently comprise a direct bond, —CH₂—, —O—, —N(H), —S—, SO₂—, —CON(H)—, —NHC(O)—, —NHCON(H)—, —NHSO₂—, —SO₂N(H)—, C(O)—O—, —NHSO₂NH—, —O—CO—, 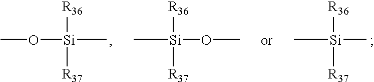
            wherein R₃₆ and R₃₇ independently comprise aryl, alkyl, -alkylene-aryl, alkoxy, or -alkyl-O-aryl;
      Y₆ comprises
         a) alkylene;
         b) alkenylene;
         c) alkynylene;
         d) arylene;
         e) heteroarylene;
         f) cycloalkylene;
         g) heterocyclylene;
         h) alkylene-arylene;
         i) alkylene-heteroarylene;
         j) alkylene-cycloalkylene;
         k) alkylene-heterocyclylene;
         l) arylene-alkylene;
         m) heteroarylene-alkylene;
         n) cycloalkylene-alkylene;
         o) heterocyclylene-alkylene;
         p) —O—;
         q) —S—;
         r) —S(O₂)—; or
         s) —S(O)—;
      wherein said alkylene groups may optionally contain one or more O, S, S(O), or SO₂ atoms;
      A₄ comprises
         a) heterocyclyl, fused arylheterocyclyl, or fused heteroarylheterocyclyl, containing at least one basic nitrogen atom,
         b) -imidazolyl, or
         c) -pyridyl; and
      R₃₃, R₃₄ and R₃₅ independently comprise hydrogen, aryl, heteroaryl, alkyl, -alkylene-aryl, or -alkylene-O-aryl; with the proviso that no two of R₃₃, R₃₄ and R₃₅ are aryl and/or heteroaryl; and R₃₃ and R₃₄ may be taken together to form a ring having the formula —(CH₂)ᵤ—X₄—(CH₂)ᵥ-bonded to the nitrogen atom to which R₃₃ and R₃₄ are attached, wherein
         u and v are, independently, 1, 2, 3, or 4;
         X₄ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 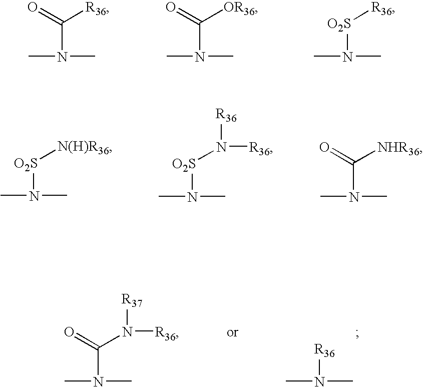
      wherein R₃₆ and R₃₇ independently comprise hydrogen, aryl, heteroaryl, alkyl, -alkylene-aryl, or -alkylene-heteroaryl; and
   wherein said ring system is optionally substituted with substituents comprising
      a) —H;
      b) -halogen;
      c) -hydroxyl;
      d) -cyano;
      e) -carbamoyl;
      f) -carboxyl;
      g) —Y₈-alkyl;
      h) —Y₈-aryl;
      i) —Y₈-heteroaryl;
      j) —Y₈-alkylene-aryl;
      k) —Y₈-alkylene-heteroaryl;
      l) —Y₈-alkylene-NR₃₈R₃₉; or
      m) —Y₈-alkylene-W₃—R₄₀;
      wherein
         Y₈ and W₃ independently comprise —CH₂—, —O—, —N(H), —S—, SO₂—, —CON(H)—, —NHC(O)—, —NHCON(H)—, —NHSO₂—, —SO₂N(H)—, C(O)—O—, —NHSO₂NH—, —O—CO—, 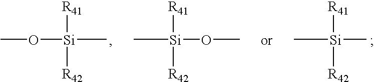
         wherein R₄₁, and R₄₂ independently comprise aryl, alkyl, -alkylene-aryl, alkoxy, or -alkyl-O-aryl; and
         R₃₈, R₃₉, and R₄₀ independently comprise hydrogen, aryl, alkyl, -alkylene-aryl, -alkylene-heteroaryl, and -alkyene-O-aryl; and R₃₈ and R₃₉ may be taken together to form a ring having the formula —(CH₂)ₙ—X₇—(CH₂)ₙ— bonded to the nitrogen atom to which R₃₈ and R₃₉ are attached wherein
            w and x are, independently, 1, 2, 3, or 4;
            X₇ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 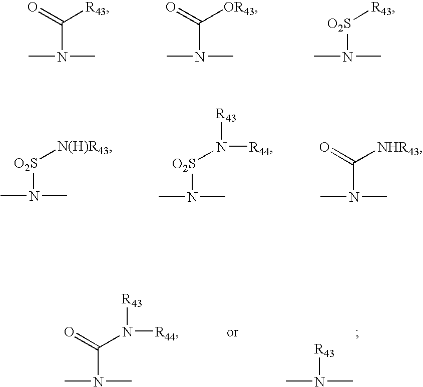
         wherein R₄₃ and R₄₄ independently comprise hydrogen, aryl, heteroaryl, alkyl, -alkylene-aryl, or -alkylene-heteroaryl;
            or a pharmaceutically acceptable salt thereof. Compounds of Formula I include Example A, Example B, Example C, and Example D, as described herein.

In another embodiment, the small molecule RAGE antagonists comprise benzimidazole compounds of Formula II. 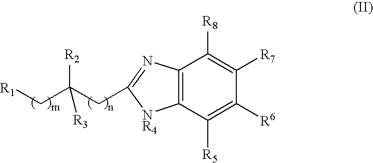
wherein for compounds of Formula (II)
m is an integer of from 0 to 3;
n is an integer of from 0 to 3;
R₁ comprises aryl;
R₂ comprises
   a) a group of the formula —N(R₉R₁₀), —NHC(O)R₉, or —NHC(O)OR₉;
   b) a group of the formula —OR₉;
   c) a group of the formula —SR₉, —SOR₉, —SO₂R₉, —SO₂NHR₉, or —SO₂N(R₉R₁₀);
   wherein R₉ and R₁₀ independently comprise
   1) —H;
   2) -Aryl;
   3) a group comprising
      a) —C₁₋₆ alkyl;
      b) —C₁₋₆ alkylaryl; 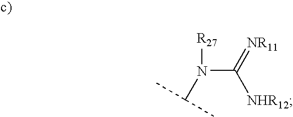
      d) -aryl;
      e) —C₁₋₆ alkyl; or
      f) —C₁₋₆ alkylaryl;
R₃ and R₄ independently comprise
   a) H;
   b) -aryl;
   c) C₁₋₆ alkyl;
   d) —C₁₋₆ alkylaryl; or
   e) —C₁₋₆ alkoxyaryl;
R₅, R₆, R₇, and R₈ independently comprise
   a) —H;
   b) —C₁₋₆ alkyl;
   c) -aryl;
   d) —C₁₋₆ alkylaryl;
   e) —C(O)—O—C₁₋₆ alkyl;
   f) —C(O)—O—C₁₋₆ alkylaryl;
   g) —C(O)—NH—C₁₋₆ alkyl;
   h) —C(O)—NH—C₁₋₆ alkylaryl;
   i) —SO₂—C₁₋₆ alkyl;
   j) —SO₂—C₁₋₆ alkylaryl;
   k) —SO₂-aryl;
   l) —SO₂—NH—C₁₋₆ alkyl;
   m) —SO₂—NH—C₁₋₆ alkylaryl;
   n) —C(O)—C₁₋₆ alkyl;
   o) —C(O)—C₁₋₆ alkylaryl;
   p)—Y—C₁₋₆ alkyl;
   q) —Y-aryl;
   r) —Y—C₁₋₆ alkylaryl;
   s) —Y—C₁₋₆ alkylene-NR₁₃R₁₄; or
   t) —Y—C₁₋₆alkylene-W—R₁₅;
      wherein Y and W independently comprise —CH₂—, —O—, —N(H)—, —S—, SO₂—, —CON(H)—, —NHC(O)—, —NHCON(H)—, —NHSO₂—, —SO₂N(H)—, —C(O)—O—, —NHSO₂NH—, —O—CO—, 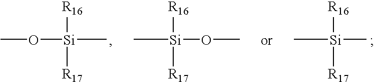
   R₁₆ and R₁₇ independently comprise aryl, C₁-C₆ alkyl, C₁-C₆ alkylaryl, C₁-C₆ alkoxy, or C₁-C₆ alkoxyaryl;
   R₁₅ independently comprise aryl, C₁-C₆ alkyl, or C₁-C₆ alkylaryl; or
   u) halogen, hydroxyl, cyano, carbamoyl, or carboxyl;
R₁₁, R₁₂, R₁₃, and R₁₄ independently comprise hydrogen, aryl, C₁-C₆ alkyl, C₁-C₆ alkylaryl, C₁-C₆ alkoxy, or C₁-C₆ alkoxyaryl;
R₁₃ and R₁₄ may be taken together to form a ring having the formula —(CH₂)ₒ—X—(CH₂)ₚ-bonded to the nitrogen atom to which R₁₃ and R₁₄ are attached, and/or R₁₁ and R₁₂ may, independently, be taken together to form a ring having the formula —CH₂)ₙ—X—(CH₂)ₚ-bonded to the atoms to which R₁₁ and R₁₂ are connected, wherein o and p are, independently, 1, 2, 3, or 4; X comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 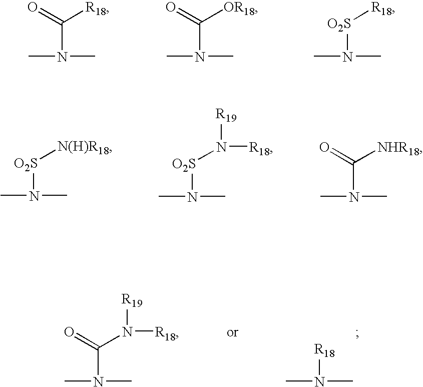
wherein the aryl and/or alkyl group(s) in R₁, R₂, R₃, R₅, R₆, R₇, R₈, R₉, R₁₀, R₁₁, R₁₂, R₁₃, R₁₄ R₁₅, R₁₆, R₁₇, R₁₈, and R₁₉ may be optionally substituted 1-4 times with a substituent group, wherein said substituent group(s) or the term substituted refers to groups comprising:
   a) —H;
   b) -Z-C₁₋₆ alkyl;
   -Z-aryl;
   -Z-C₁₋₆ alkylaryl;
   -Z-C₁₋₆-alkyl-NR₂₀R₂₁;
   -Z-C₁₋₆-alkyl-W—R₂₂;
      wherein Z and W independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—, 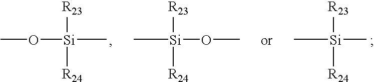
      wherein;
      R₂₀ and R₂₁ independently comprise hydrogen, aryl, C₁-C₆ alkyl, C₁-C₆ alkylaryl, C₁-C₆ alkoxy, or C₁-C₆ alkoxyaryl;
      R₂₂, R₂₃, and R₂₄ independently comprise aryl, C₁-C₆ alkyl, C₁-C₆ alkylaryl, C₁-C₆ alkoxy, or C₁-C₆ alkoxyaryl; or
   c) halogen, hydroxyl, cyano, carbamoyl, or carboxyl; and
R₂₀ and R₂₁ may be taken together to form a ring having the formula —(CH₂)_{q}—X—(CH₂)ᵣ-bonded to the nitrogen atom to which R₂₀ and R₂, are attached wherein q and r are, independently, 1, 2, 3, or 4; X comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 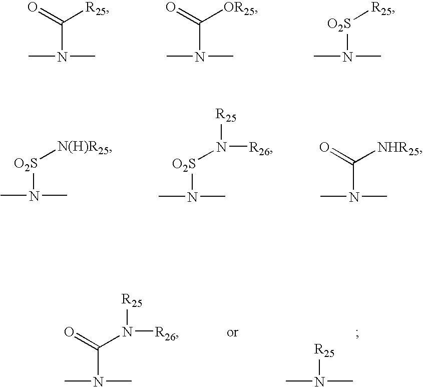
R₂₅, R₂₆, and R₂₇ independently comprise hydrogen, aryl, C₁-C₆ alkyl, or C₁-C₆ alkylaryl; or a pharmaceutically acceptable salt, solvate or prodrug thereof.

In yet another embodiment, the small molecule RAGE antagonist comprises carboxamide compounds of Formula (III): 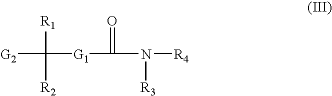
wherein for compound of Formula (III)
G₁ comprises C₁-C₆ alkylene or (CH₂)ₖ, where k is 0 to 3;
G₂ comprises
   a) hydrogen
   b) —C₁₋₆ C₁₋₆ alkyl;
   c) -aryl;
   d) —C₁₋₆ alkylaryl 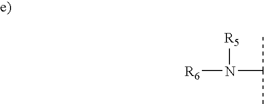
   where R₅ and R₆ independently comprise
      i) —H;
      ii)-C₁₋₆ alkyl;
      iii) -aryl;
      iv) —C₁₋₆ alkylaryl;
      v) —C(O)—O—C₁₋₆ alkyl;
      vi) —C(O)—O—C₁₋₆ alkylaryl;
      vii) —C(O)—O—C₁₋₆ alkylcycloalkylaryl;
      viii) —C(O)—NH—C₁₋₆ alkyl;
      ix) —C(O)—NH—C₁₋₆ alkylaryl;
      x) —SO₂—C₁₋₆ alkyl;
      xi) —SO₂—C₁₋₆ alkylaryl;
      xii) —SO₂-aryl;
      xiii) —SO₂—NH—C₁₋₆ alkyl;
      xiv) —SO₂—NH—C₁₋₆ alkylaryl; 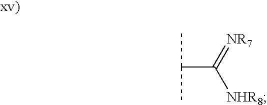
      xvi) —C(O)—C₁₋₆ alkyl; or
      xvii) —C(O)—C₁₋₆ alkylaryl; or
   f) a group of the formula 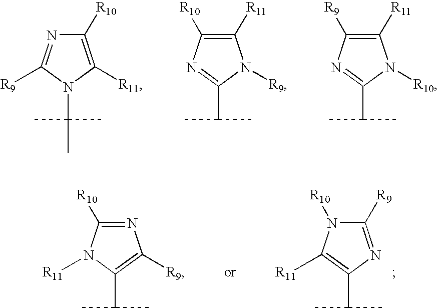
   wherein
      R₉, R₁₀, and R₁₁ may comprise hydrogen; or
      R₉, R₁₀, and R₁₁ independently comprise
      i) —C₁₋₆ alkyl;
      ii) -aryl;
      iii) —C₁₋₆ alkylaryl;
      iv) —C(O)—O—C₁₋₆ alkyl;
      v) —C(O)—O—C₁₋₆ alkylaryl;
      vi) —C(O)—NH—C₁₋₆ alkyl;
      vii) —C(O)—NH—C₁₋₆ alkylaryl;
      viii) —SO₂—C₁₋₆ alkyl;
      ix) —SO₂—C₁₋₆ alkylaryl;
      x) —SO₂-aryl;
      xi) —SO₂—NH—C₁₋₆ alkyl;
      xii) —SO₂—NH—C₁₋₆ alkylaryl;
      xiii) —C(O)—C₁₋₆ alkyl; or
      xiv) —C(O)—C₁₋₆ alkylaryl;
   or R₁₀ and R₁₁ may be taken together to constitute a fused cycloalkyl, fused heterocyclyl, or fused aryl ring containing the atoms to which R₁₀ and R₁₁ are bonded;
R₁ comprises
   a) hydrogen;
   b) —C₁₋₆ alkyl;
   c) -aryl; or
   d) —C₁₋₆ alkylaryl;
R₂ comprises
   a) —C₁₋₆ alkyl;
   b) -aryl;
   c) —C₁₋₆ alkylaryl; or
   d) a group of the formula 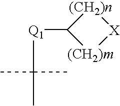
wherein m and n are independently selected from 1, 2, 3, or 4; X comprises a direct bond, CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 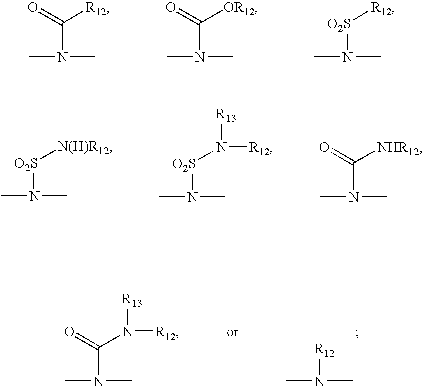
   -Q₁- comprises C₁₋₆ alkylene, C₂₋₆ alkenylene, or C₂₋₆ alkynylene;
R₃ comprises
   a) hydrogen;
   b) —C₁₋₆ alkyl;
   c) —C₁₋₆ alkylaryl; or
   d) —C₁₋₆ alkoxyaryl;
R₄ comprises
   a) —C₁₋₆ alkylaryl;
   b) —C₁₋₆ alkoxyaryl; or
   c) -aryl;
R₇, R₈, R₁₂ and R₁₃ independently comprise hydrogen, C₁-C₆ alkyl, C₁-C₆ alkylaryl, or aryl; and wherein
the aryl and/or alkyl group(s) in R₁, R₂, R₃, R₄, R₅, R₆, R₇, R₈, and R₉, R₁₀, R₁₁, and R₁₂, and R₁₃ may be optionally substituted 1-4 times with a substituent group, wherein said substituent group(s) or the term substituted refers to groups comprising:
   a) —H;
   b) —Y—C₁₋₆ alkyl;
   —Y-aryl;
   —Y—C₁₋₆ alkylaryl;
   —Y—C₁₋₆-alkyl-NR₁₄R₁₅;
   —Y—C₁₋₆-alkyl-W—R₁₆;
      wherein Y and W independently comprise —CH₂—, —O—, —N(H), —S—, SO₂—, —CON(H)—, —NHC(O)—, —NHCON(H)—, —NHSO₂—, —SO₂N(H)—, —C(O)—O—, —N—HSO₂NH—, —O—CO—, 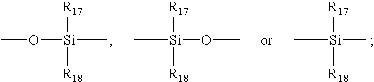
      R₁₆, R₁₇ and R₁₈ comprise hydrogen, aryl, C₁-C₆ alkyl, C₁-C₆ alkylaryl, C₁-C₆ alkoxy, or C₁-C₆ alkoxyaryl; or
   c) halogen, hydroxyl, cyano, carbamoyl, or carboxyl; and
R₁₄ and R₁₅ independently comprise hydrogen, aryl, C₁-C₆ alkyl, or C₁-C₆ alkylaryl; and wherein
R₁₄ and R₁ may be taken together to form a ring having the formula —(CH₂)ₒ-Z-(CH₂)ₚ-bonded to the nitrogen atom to which R₁₄ and R₁₅ are attached, and/or R₇ and R₈ may, independently, be taken together to form a ring having the formula —(CH₂)ₒ-Z-(CH₂)ₚ-bonded to the atoms to which R₇ and R₈ are attached, wherein o and p are, independently, 1, 2, 3, or 4; Z comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 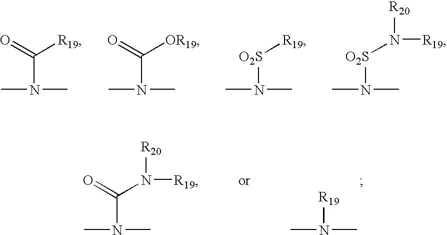
R₁₉ and R₂₀ independently comprise hydrogen, aryl, C₁-C₆ alkyl, or C₁-C₆ alkylaryl.

The present invention also comprises compounds of Formula (IV) as small molecule RAGE antagonists capable of reversing Alzheimer's Disease: 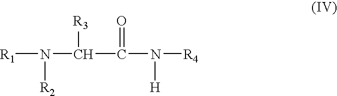
wherein for compounds of Formula (IV):
R₁ and R₂ are independently selected from
   a) —H;
   b) —C₁₋₆ alkyl;
   c) -aryl;
   d) —C₁₋₆ alkylaryl;
   e) —C(O)—O—C₁₋₆ alkyl;
   f) —C(O)—O—C₁₋₆ alkylaryl;
   g) —C(O)—NH—C₁₋₆ alkyl;
   h) —C(O)—NH—C₁₋₆ alkylaryl;
   i) —SO₂—C₁₋₆ alkyl;
   j) —SO₂—C₁₋₆ alkylaryl;
   k) —SO₂-aryl;
   l) —SO₂—NH—C₁₋₆ alkyl;
   m) —SO₂—NH—C₁₋₆ alkylaryl;
   n) 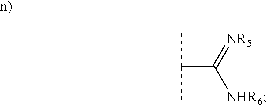
   o) —C(O)—C₁₋₆ alkyl; and
   p) —C(O)—C₁₋₆ alkylaryl;
R₃ is selected from
   a) —C₁₋₆ alkyl;
   b) -aryl; and
   c) —C₁₋₆ alkylaryl;
R₄ is selected from
   a) —C₁₋₆ alkylaryl;
   b) —C₁₋₆ alkoxyaryl; and
   c) -aryl;
R₅ and R₆ are independently selected from the group consisting of hydrogen, C₁-C₆ alkyl, C₁-C₆ alkylaryl, and aryl; and wherein
the aryl and/or alkyl group(s) in R₁, R₂, R₃, R₄, R₅, R₆, R₇, R₈, R₉, R₁₀, R₁₈, R₁₉, and R₂₀ may be optionally substituted 1-4 times with a substituent group, wherein said substituent group(s) or the term substituted refers to groups selected from the group consisting of:
   a) —H;
   b) —Y—C₁₋₆ alkyl;
   —Y-aryl;
   —Y—C₁₋₆ alkylaryl;
   —Y—C₁₋₆-alkyl-NR₇R₈; and
   —Y—C₁₋₆-alkyl-W—R₂₀;
      wherein Y and W are, independently selected from the group consisting of —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—, 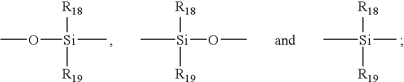
      and
   c) halogen, hydroxyl, cyano, carbamoyl, or carboxyl; and
R₁₈ and R₁₉ are independently selected from the group consisting of aryl, C₁-C₆ alkyl, C₁-C₆ alkylaryl, C₁-C₆ alkoxy, and C₁-C₆ alkoxyaryl;
R₂₀ is selected from the group consisting of aryl, C₁-C₆ alkyl, and C₁-C₆ alkylaryl;
R₇, R₈, R₉ and R₁₀ are independently selected from the group consisting of hydrogen, aryl, C₁-C₆ alkyl, and C₁-C₆ alkylaryl; and wherein
R₇ and R₈ may be taken together to form a ring having the formula —(CH₂)ₘ—X—(CH₂)ₙ— bonded to the nitrogen atom to which R₇ and R₈ are attached, and/or R₅ and R₆ may, independently, be taken together to form a ring having the formula —(CH₂)ₘ—X—(CH₂)ₙ— bonded to the nitrogen atoms to which R₅ and R₆ are attached, wherein m and n are, independently, 1, 2, 3, or 4; X is selected from the group consisting of —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 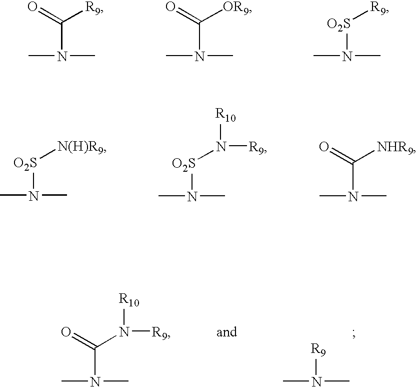
   a pharmaceutically acceptable salt, solvate or prodrug thereof.

In the compounds of Formula (I), (II), (III) and (IV), the various functional groups represented should be understood to have a point of attachment at the functional group having the hyphen. In other words, in the case of —C₁₋₆ alkylaryl, it should be understood that the point of attachment is the alkyl group; an example would be benzyl. In the case of a group such as —C(O)—NH—C₁₋₆ alkylaryl, the point of attachment is the carbonyl carbon.

Also included within the scope of the invention are the individual enantiomers of the compounds represented by Formulas (I), (II), (III), and (IV) above as well as any wholly or partially racemic mixtures thereof. The present invention also covers the individual enantiomers of the compounds represented by the Formulas above as mixtures with diastereoisomers thereof in which one or more stereocenters are inverted.

Definitions

As used herein, “RAGE”, encompasses a peptide which has the full amino acid sequence of RAGE as shown in Neeper et al., J. Biol. Chem., 267:15998-15004 (1992) or a polypeptide having conservative amino acid substitutions or deletions, wherein conservative amino acid substitutions or deletions are those alterations which do not significantly effect the structure or function of the peptide. Also, as used herein, a “fragment” of RAGE is at least 5 amino acids in length, preferably more than 15 amino acids in length, but is less than the full length shown in Neeper et al., (1992).

As defined herein, compounds that prevent or antagonize the binding of ligands to RAGE are RAGE antagonists. As described above, RAGE antagonists of the present invention may comprise small molecule RAGE antagonists, such as the compounds of Formulas (I), (II), (III), and (IV) as well as polypeptides, such as sRAGE and the RAGE V-domain, or fragments thereof. The peptides may be modified to increase their stability in vivo. For example, in an embodiment, the peptides may comprise conservative substitutions, wherein conservative amino acid substitutions are those substitutions which do not significantly effect the structure or function of the peptide. Also, the polypeptide may be a non-natural polypeptide which has chirality not found in nature, i.e., D-amino acids in place of L-amino acids.

As used herein, a peptidomimetic compound has a bond, a peptide backbone or an amino acid component replaced with a suitable mimic. Examples of unnatural amino acids which may be suitable amino acid mimics include (β-alanine, L-α-amino butyric acid, L-γ-amino butyric acid, L-α-amino isobutyric acid, L-ε-amino caproic acid, L-aspartic acid, L-glutamic acid, N-ε-Boc-N-α-CBZ-L-lysine, L-norleucine, L-norvaline, Boc-p-nitro-L-phenylalanine, Boc-hydroxyproline, and the like (WO 01/12598).

In another embodiment, the RAGE antagonist is an antibody to RAGE. In one embodiment, the antibody is a monoclonal antibody. The monoclonal antibody may be human, humanized, primatized, or a chimeric antibody. In yet another embodiment, the RAGE antagonist is a fragment of an antibody. For example, the RAGE antagonist may comprise a Fab fragment of an anti-RAGE antibody. Preferably, the Fab fragment is a F(ab′)Z fragment. In an embodiment, the above compound comprises the variable domain of an anti-RAGE antibody. In an embodiment, the antibody is an IgG antibody.

Chemical terms used to describe small molecule RAGE antagonists of Formulas (I), (II), (III), and (IV) are described below.

As used herein, the term “lower” refers to a group having between one and six carbons.

As used herein, the term “alkyl” refers to a straight or branched chain hydrocarbon having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ atoms. Examples of “alkyl” as used herein include, but are not limited to, methyl, n-butyl, t-butyl, n-pentyl, isobutyl, and isopropyl, and the like.

As used herein, the term “alkylene” refers to a straight or branched chain divalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ atoms. Examples of “alkylene” as used herein include, but are not limited to, methylene, ethylene, and the like.

As used herein, the term “alkyline” refers to a straight or branched chain trivalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “alkyline” as used herein include, but are not limited to, methine, 1,1,2-ethyline, and the like.

As used herein, the term “alkenyl” refers to a hydrocarbon radical having from two to ten carbons and at least one carbon—carbon double bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may containing one or more O, S, S(O), or S(O)₂ atoms.

As used herein, the term “alkenylene” refers to a straight or branched chain divalent hydrocarbon radical having from two to ten carbon atoms and one or more carbon—carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)₂ atoms. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from two to ten carbons and at least one carbon—carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ atoms.

As used herein, the term “alkynylene” refers to a straight or branched chain divalent hydrocarbon radical having from two to ten carbon atoms and one or more carbon—carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)₂ atoms. Examples of “alkynylene” as used herein include, but are not limited to, ethyne-1,2-diyl, propyne-1,3-diyl, and the like.

As used herein, “cycloalkyl” refers to an alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “heterocyclic” or the term “heterocyclyl” refers to a three to twelve-membered heterocyclic ring optionally possessing one or more degrees of unsaturation, containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, 1,4-dioxane, 1,3-dioxane, piperidine, pyrrolidine, morpholine, piperazine, and the like.

As used herein, the term “heterocyclyl containing at least one basic nitrogen atom” refers to a “heterocyclic” “heterocyclyl” group as defined above, wherein said heterocyclyl group contains at least one nitrogen atom flanked by hydrogen, alkyl, alkylene, or alkylyne groups, wherein said alkyl and/or alkylene groups are not substituted by oxo. Examples of “heterocyclyl containing at least one basic nitrogen atom” include, but are not limited to, piperazine-2-yl, pyrrolidine-2-yl, azepine-4-yl, 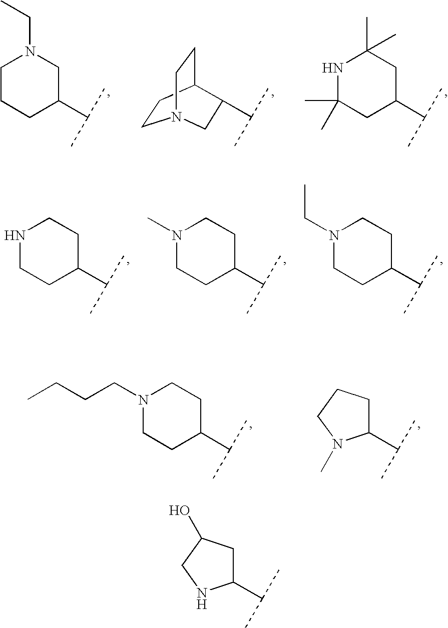
and the like.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-diyl, and the like.

As used herein, the term “aryl” refers to a benzene ring or to an optionally substituted benzene ring system fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy optionally substituted by acyl, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-naphthyl, 1-naphthyl, 1-anthracenyl, and the like.

As used herein, the term “arylene” refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

As used herein, the term “heteroaryl” refers to a five- to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “fused cycloalkylaryl” refers to one or more cycloalkyl groups fused to an aryl group, the aryl and cycloalkyl groups having two atoms in common, and wherein the aryl group is the point of substitution. Examples of “fused cycloalkylaryl” used herein include 5-indanyl, 5,6,7,8-tetrahydro-2-naphthyl, 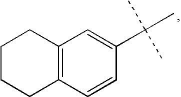
and the like.

As used herein, the term “fused cycloalkylarylene” refers to a fused cycloalkylaryl, wherein the aryl group is divalent. Examples include 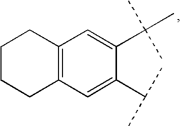
and the like.

As used herein, the term “fused arylcycloalkyl” refers to one or more aryl groups fused to a cycloalkyl group, the cycloalkyl and aryl groups having two atoms in common, and wherein the cycloalkyl group is the point of substitution. Examples of “fused arylcycloalkyl” used herein include 1-indanyl, 2-indanyl, 9-fluorenyl, 1-(1,2,3,4-tetrahydronaphthyl), 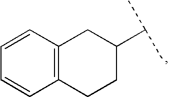
and the like.

As used herein, the term “fused arylcycloalkylene” refers to a fused arylcycloalkyl, wherein the cycloalkyl group is divalent. Examples include 9,1-fluorenylene, 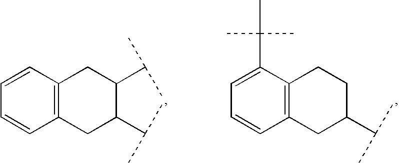
and the like.

As used herein, the term “fused heterocyclylaryl” refers to one or more heterocyclyl groups fused to an aryl group, the aryl and heterocyclyl groups having two atoms in common, and wherein the aryl group is the point of substitution. Examples of “fused heterocyclylaryl” used herein include 3,4-methylenedioxy-1-phenyl, 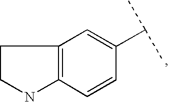
and the like

As used herein, the term “fused heterocyclylarylene” refers to a fused heterocyclylaryl, wherein the aryl group is divalent. Examples include 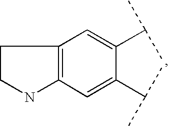
and the like.

As used herein, the term “fused arylheterocyclyl” refers to one or more aryl groups fused to a heterocyclyl group, the heterocyclyl and aryl groups having two atoms in common, and wherein the heterocyclyl group is the point of substitution. Examples of “fused arylheterocyclyl” used herein include 2-(1,3-benzodioxolyl), 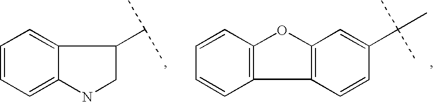
and the like.
As used herein, the term “fused arylheterocyclyl containing at least one basic nitrogen atom” refers to a “fused arylheterocyclyl” group as defined above, wherein said heterocyclyl group contains at least one nitrogen atom flanked by hydrogen, alkyl, alkylene, or alkylyne groups, wherein said alkyl and/or alkylene groups are not substituted by oxo. Examples of “fused arylheterocyclyl containing at least one basic nitrogen atom” include, but are not limited to, 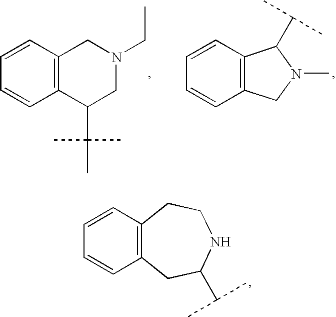
and the like.

As used herein, the term “fused arylheterocyclylene” refers to a fused arylheterocyclyl, wherein the heterocyclyl group is divalent. Examples include 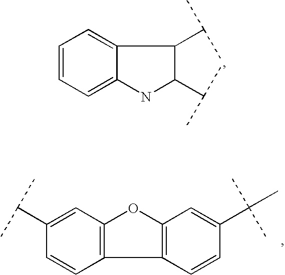
and the like.

As used herein, the term “fused cycloalkylheteroaryl” refers to one or more cycloalkyl groups fused to a heteroaryl group, the heteroaryl and cycloalkyl groups having two atoms in common, and wherein the heteroaryl group is the point of substitution. Examples of “fused cycloalkylheteroaryl” used herein include 5-aza-6-indanyl, 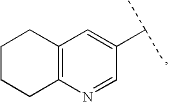
and the like.

As used herein, the term “fused cycloalkylheteroarylene” refers to a fused ycloalkylheteroaryl, wherein the heteroaryl group is divalent. Examples include 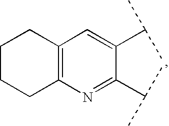
and the like.

As used herein, the term “fused heteroarylcycloalkyl” refers to one or more heteroaryl groups fused to a cycloalkyl group, the cycloalkyl and heteroaryl groups having two atoms in common, and wherein the cycloalkyl group is the point of substitution. Examples of “fused heteroarylcycloalkyl” used herein include 5-aza-1-indanyl, 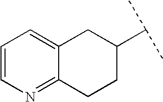
and the like.

As used herein, the term “fused heteroarylcycloalkylene” refers to a fused heteroarylcycloalkyl, wherein the cycloalkyl group is divalent. Examples include 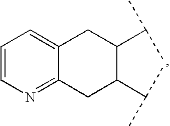
and the like.

As used herein, the term “fused heterocyclylheteroaryl” refers to one or more heterocyclyl groups fused to a heteroaryl group, the heteroaryl and heterocyclyl groups having two atoms in common, and wherein the heteroaryl group is the point of substitution. Examples of “fused heterocyclylheteroaryl” used herein include 1,2,3,4-tetrahydro-beta-carbolin-8-yl, 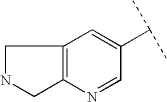
and the like.

As used herein, the term “fused heterocyclylheteroarylene” refers to a fused heterocyclylheteroaryl, wherein the heteroaryl group is divalent. Examples include 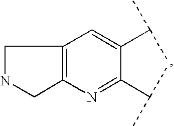
and the like.

As used herein, the term “fused heteroarylheterocyclyl” refers to one or more heteroaryl groups fused to a heterocyclyl group, the heterocyclyl and heteroaryl groups having two atoms in common, and wherein the heterocyclyl group is the point of substitution. Examples of “fused heteroarylheterocyclyl” used herein include -5-aza-2,3-dihydrobenzofuran-2-yl, 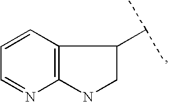
and the like.
As used herein, the term “fused heteroarylheterocyclyl containing at least one basic nitrogen atom” refers to a “fused heteroarylheterocyclyl” group as defined above, wherein said heterocyclyl group contains at least one nitrogen atom flanked by hydrogen, alkyl, alkylene, or alkylyne groups, wherein said alkyl and/or alkylene groups are not substituted by oxo. Examples of “fused heteroarylheterocyclyl containing at least one basic nitrogen atom” include, but are not limited to, 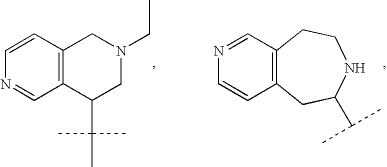
and the like.

As used herein, the term “fused heteroarylheterocyclylene” refers to a fused heteroarylheterocyclyl, wherein the heterocyclyl group is divalent. Examples include 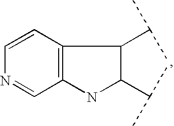
and the like.

As used herein, the term “acid isostere” refers to a substituent group which will ionize at physiological pH to bear a net negative charge. Examples of such “acid isosteres” include but are not limited to heteroaryl groups such as but not limited to isoxazol-3-ol-5-yl, 1H-tetrazole-5-yl, or 2H-tetrazole-5-yl. Such acid isosteres include but are not limited to heterocyclyl groups such as but not limited to imidazolidine-2,4-dione-5-yl, imidazolidine-2,4-dione-1-yl, 1,3-thiazolidine-2,4-dione-5-yl, or 5-hydroxy-4H-pyran-4-on-2-yl.

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “alkoxy” refers to the group RₐO—, where Rₐ is alkyl.

As used herein, the term “alkenyloxy” refers to the group RₐO—, where Rₐ is alkenyl.

As used herein, the term “alkynyloxy” refers to the group RₐO—, where Rₐ is alkynyl.

As used herein, the term “alkylsulfanyl” refers to the group RₐS—, where Rₐ is alkyl.

As used herein, the term “alkenylsulfanyl” refers to the group RₐS—, where Rₐ is alkenyl.

As used herein, the term “alkynylsulfanyl” refers to the group RₐS—, where Rₐ is alkynyl.

As used herein, the term “alkylsulfenyl” refers to the group RₐS(O)—, where Rₐ is alkyl.

As used herein, the term “alkenylsulfenyl” refers to the group RₐS(O)—, where Rₐ is alkenyl.

As used herein, the term “alkynylsulfenyl” refers to the group RₐS(O)—, where Rₐ is alkynyl.

As used herein, the term “alkylsulfonyl” refers to the group RₐSO₂—, where Rₐ is alkyl.

As used herein, the term “alkenylsulfonyl” refers to the group RₐSO₂—, where Rₐ is alkenyl.

As used herein, the term “alkynylsulfonyl” refers to the group RₐSO₂—, where Rₐ is alkynyl.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “alkoxycarbonyl” refers to the group RₐOC(O)—, where Rₐ is alky, alkenyl, alkynyl, cycloalkyl, cycloalkenyl, or heterocyclyl.

As used herein, the term “aryloxycarbonyl” refers to the group RₐOC(O)—, where Rₐ is aryl or heteroaryl.

As used herein, the term “aroyloxy” refers to the group RₐC(O)O—, where Rₐ is aryl.

As used herein, the term “heteroaroyloxy” refers to the group RₐC(O)O—, where Rₐ is heteroaryl.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Alkyl or cycloalkyl substituents shall be recognized as being functionally equivalent to those having one or more degrees of unsaturation. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” shall include iodine, bromine, chlorine and fluorine.

As used herein, the term “mercapto” shall refer to the substituent —SH.

As used herein, the term “carboxy” shall refer to the substituent —COOH.

As used herein, the term “cyano” shall refer to the substituent —CN.

As used herein, the term “aminosulfonyl” shall refer to the substituent —SO₂NH₂.

As used herein, the term “carbamoyl” shall refer to the substituent —C(O)NH₂.

As used herein, the term “sulfanyl” shall refer to the substituent —S—.

As used herein, the term “sulfenyl” shall refer to the substituent —S(O)—.

As used herein, the term “sulfonyl” shall refer to the substituent —S(O)₂—.

As used herein, the term “solvate” is a complex of variable stoichiometry formed by a solute (in this invention, a compound of Formula (I), (II), (III), or (IV)) and a solvent. Such solvents for the purpose of the invention may not interfere with the biological activity of the solute. Solvents may be, by way of example, water, ethanol, or acetic acid.

As used herein, the term “biohydrolyzable ester” is an ester of a drug substance (in this invention, a compound of Formula (I), (II), (III), or (IV)) which either: (a) does not interfere with the biological activity of the parent substance but confers on that substance advantageous properties in vivo such as duration of action, onset of action, and the like; or (b) is biologically inactive but is readily converted in vivo by the subject to the biologically active principle. The advantage is that, for example, the biohydrolyzable ester is orally absorbed from the gut and is transformed to a compound of Formula (I), (II), (III), or (IV) in plasma. Many examples of such are known in the art and include by way of example lower alkyl esters (e.g., C₁-C₄), lower acyloxyalkyl esters, lower alkoxyacyloxyalkyl esters, alkoxyacyloxy esters, alkyl acylamino alkyl esters, and choline esters.

As used herein, the term “biohydrolyzable amide” is an amide of a drug substance (in this invention, a compound of general Formula (I), (II), (III), or (IV)) which either: (a) does not interfere with the biological activity of the parent substance but confers on that substance advantageous properties in vivo such as duration of action, onset of action, and the like; or (b) is biologically inactive but is readily converted in vivo by the subject to the biologically active principle. The advantage is that, for example, the biohydrolyzable amide is orally absorbed from the gut and is transformed to (I), (II), (III), or (IV) in plasma. Many examples of such are known in the art and include by way of example lower alkyl amides, α-amino acid amides, alkoxyacyl amides, and alkylaminoalkylcarbonyl amides.

As used herein, the term “prodrug” includes biohydrolyzable amides and biohydrolyzable esters and also encompasses: (a) compounds in which the biohydrolyzable functionality in such a prodrug is encompassed in the compound of Formula (I), (II), (III), or (IV): for example, the lactam formed by a carboxylic group and an amine; and (b) compounds which may be oxidized or reduced biologically at a given functional group to yield drug substances of Formula (I), (II), (III), or (IV). Examples of these functional groups include, but are not limited to, 1,4-dihydropyridine, N-alkylcarbonyl-1,4-dihydropyridine, 1,4-cyclohexadiene, tert-butyl, and the like.

The term “pharmacologically effective amount” or shall mean that amount of a drug or pharmaceutical agent that will elicit the biological or medical response of a tissue, animal or human that is being sought by a researcher or clinician. This amount can be a therapeutically effective amount. The term “therapeutically effective amount” shall mean that amount of a drug or pharmaceutical agent that will elicit the therapeutic response of an animal or human that is being sought.

The term “treatment” or “treating” as used herein, refers to the full spectrum of treatments for a given disorder from which the patient is suffering, including alleviation of one, most of all symptoms resulting from that disorder, to an outright cure for the particular disorder or prevention of the onset of the disorder.

RAGE Antagonists and Amyloidosis

The present invention comprises the use of antagonists for the Receptor for Advanced Glycation Endproducts (RAGE) to reverse pre-existing amyloidosis and the symptoms thereof. Additionally, the present invention comprises the use of small molecule RAGE antagonists to inhibit the onset of amyloid plaque formation and to prevent the symptoms thereof.

The Receptor for Advanced Glycated Endproducts (RAGE) is a member of the immunoglobulin super family of cell surface molecules. RAGE is a receptor for Advanced Glycation Endproducts (AGEs). AGEs are derived from the nonenzymatic glycation and oxidation of amino groups on proteins to form Amadori adducts, which may undergo additional rearrangements, dehydrations, and cross-linking with other proteins to form AGEs.

The extracellular (N-terminal) domain of RAGE includes three immunoglobulin-type regions: one V (variable) type domain followed by two C-type (constant) domains (Neeper et al., J. Biol. Chem., 267:14998-15004 (1992)). A single transmembrane spanning domain and a short, highly charged cytosolic tail follow the extracellular domain. The N-terminal, extracellular domain can be isolated by proteolysis of RAGE to generate soluble RAGE (sRAGE) comprised of the V and C domains.

RAGE is expressed in most tissues, and in particular, is found in cortical neurons during embryogenesis (Hori et al., J. Biol. Chem., 270:25752-761 (1995)). Increased levels of RAGE are also found in aging tissues (Schleicher et al., J. Clin. Invest., 99 (3): 457-468 (1997)), and the diabetic retina, vasculature and kidney (Schmidt et al., Nature Med., 1:1002-1004 (1995)). Activation of RAGE in different tissues and organs leads to a number of pathophysiological consequences. RAGE has been implicated in a variety of conditions including: acute and chronic inflammation (Hofmann et al., Cell, 97:889-901 (1999)), the development of diabetic late complications such as increased vascular permeability (Wautier et al., J. Clin. Invest., 97:238-243 (1995)), nephropathy (Teillet et al., J. Am. Soc. Nephrol., 11:1488-1497 (2000)), atherosclerosis (Vlassara et. al., The Finnish Medical Society DUODECIM, Ann. Med., 28:419-426 (1996)), and retinopathy (Hammes et al., Diabetologia, 42:603-607 (1999)). RAGE has also been implicated in Alzheimer's disease (Yan et al., Nature, 382: 685-691, (1996)), erectile dysfunction, and in tumor invasion and metastasis (Taguchi et al., Nature, 405: 354-357, (2000)).

In addition to Advanced Glycation Endproducts (AGEs), other compounds can bind to, and modulate RAGE. Thus, RAGE interacts with amphoterin, a polypeptide which mediates neurite outgrowth in cultured embryonic neurons (Hori et al., J. Biol. Chem., 270:25752-761 (1995)), and EN-RAGE, a protein having substantial similarity to calgranulin (Hofmann et al., Cell, 97:889-901 (1999)). RAGE has also been shown to interact with β-amyloid (Yan et al., Nature, 389:589-595, (1997); Yan et al., Nature, 382:685-691 (1996); Yan et al., Proc. Natl. Acad. Sci., 94:5296-5301 (1997)).

Amyloidosis is a diverse group of disease processes characterized by extracellular tissue deposits of amyloid proteins. Amyloid is distinguished by a starch-like staining reaction with iodine, characteristic tinctorial and optical properties upon exposure to Congo red, a distinctive protein fibril structure, and an extracellular distribution.

Amyloid deposition may be a primary disease, or may be secondary to another pathological condition. Primary amylodosis tends to affect mesodermal tissues, such as peripheral nerves, skin, tongue, joints, and liver. Secondary amyloidosis mainly affects parenchymatous organs, such as spleen, liver, kidneys and adrenals. Many diseases are associated with abnormal α-amyloid proteins. For example, AL (amyloid light chain) amyloidosis comprises a defect in the immunoglobulin light chain and occurs in primary amyloidosis and in amyloidosis associated with multiple myeloma. In contrast, AA (amyloid associated) amyloidosis has a nonimmunoglobulin protein made from the serum precursor SAA. This form of amyloidosis occurs primarily as a complication of long-standing inflammatory diseases.

Other diseases are caused by additional amyloid proteins, such as amyloid-β (Aβ, which appears to play a role in Alzheimer's Disease (AD). Aβ is a 39-43 residue polypeptide derived by proteolytic processing of βAPP. Aβ forms a spectrum of macromolecular assemblies, ranging from monomer and dimer to complex aggregates and Congophilic fibrils (Pike et al., Neurosci. 13:1676-1687 (1993); Haass et al., Cell, 75:1039-1042 (1993)).

AGEs, the natural ligand for RAGE, have no apparent structural similarity to Aβ. However, the binding of RAGE to either AGEs or Aβ appears to result from specific molecular interactions. RAGE binds Advanced Glycation Endproducts (AGEs) and Aβ principally via determinants in the V-domain, and triggers signal transduction mechanisms following engagement of cytosolic proteins. Although AGEs bind RAGE, there is no binding of similarly derivatized proteins, such as oxidized lipoproteins or formylated or maleylated albumin to RAGE (Yan et al., J. Biol. Chem., 269:9889-9897 (1994)). Similarly, Aβ (1-40/42) binds to RAGE, but scrambled Aβ (1-40) and multiple unrelated peptides, including those with similar content of random or β-sheet structures, do not bind to RAGE.

RAGE may also serve as a target for amyloidogenic proteins/peptides or fibrils by interactions with fibrillar serum amyloid A (SAA) proteins, amylin, prion peptides and transthyretin (Yan et al., Nat. Med., 6:643-651 (2000); Sousa et al., Lab. Invest., 80:1101-1110 (2000); WO 01/12598). RAGE appears to bind β-sheet fibrillar material regardless of the composition of the subunits (amyloid-β peptide, Aβ, amylin, serum amyloid A, prion-derived peptide) (Yan, S.-D., et al., Nature, 382:685-691 (1996); Yan, S-D., et al., Nat. Med., 6:643-651 (2000)). Thus, RAGE may serve as a focal point for fibril assembly, with binding of fibrils to RAGE contributing to RAGE-mediated activation of the MAP kinase pathway. For example, blocking RAGE with specific antibodies or using a soluble form of RAGE (sRAGE) inhibits the interaction of fibrils with the receptor and may attenuate systemic amyloidosis measured as deposition of α-amyloid (SAA) in plasma and spleen (WO 01/12598). Additionally, other studies indicate that Aβ binding to RAGE at the brain endothelium in vivo may increase transport of circulating Aβ into the central nervous system, and that this transport may be inhibited using sRAGE and anti-RAGE IgG (US 2002/0116725), and that Aβ binding to RAGE may cause activation of RAGE-mediated cellular activation (WO 97/26913). Thus, sRAGE and anti-RAGE antibodies may inhibit the binding of Aβ to RAGE as well as some aspects of RAGE-induced cellular stress (WO 97/26913).

Also, deposition of amyloid may result in enhanced expression of RAGE. For example, in the brains of patients with Alzheimer's disease (AD), RAGE expression increases in neurons and glia (Yan, S.-D., et al., Nature 382:685-691 (1996)). However, the consequences of AB interaction with RAGE appear to be quite different in neurons versus microglia. Whereas microglia become activated as a consequence of AB-RAGE interaction, as reflected by increased motility and expression of cytokines, early RAGE-mediated neuronal activation is superceded by cytotoxicity at later times.

Rage Antagonists Reduce Plaque Volume

Although studies suggested that the interaction of Aβ peptides and/or amyloid-containing fibrils with RAGE may trigger subsequent molecular events at the cellular level, there have been no studies which have investigated the ability of RAGE antagonists to reduce plaque formation at the macromolecular level once the plaques have already formed. The present invention recognizes that RAGE antagonists can be used in vivo as therapeutics to inhibit amyloid plaque formation, and to reduce the size of pre-existing amyloid plaques. In an embodiment, reversal of plaque size is associated with a reversal of the cognitive loss associated with Alzheimer's disease.

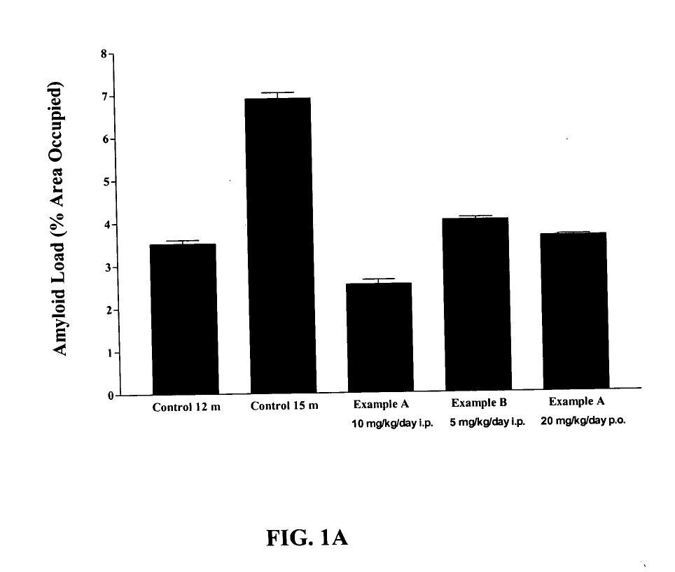

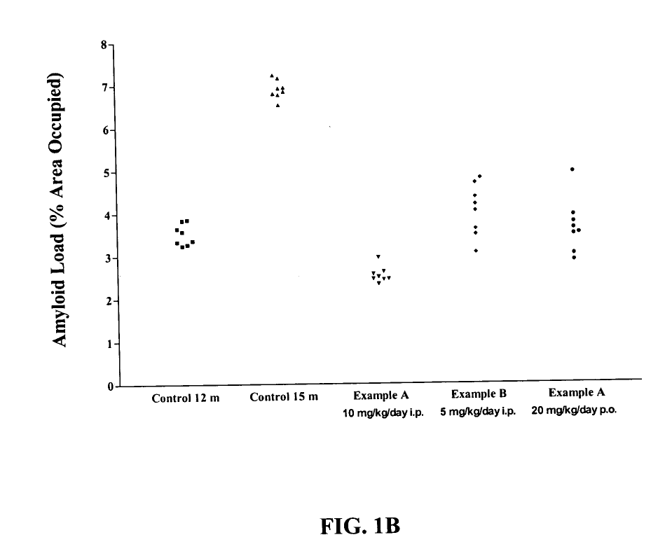

For example, as shown in FIG. 1, intraperitoneal injection or oral administration of RAGE antagonists Example A and Example B reduces plaque formation in an APP transgenic mouse model of established, later-stage Alzheimer's Disease (AD). In the APP transgenic mouse model, AD begins to develop by about 6 months. Intraperitoneal (i.p.) injection of Example A at a dose of 10 mg/kg per day, or of Example B at a dose of 5 mg/kg/day into 12 month old APP transgenic mice for 3 months (i.e., until 15 months) reduces plaque formation as compared to age-matched AD mice injected with saline (FIG. 1, panels A and B, compare 15 month (15 m) control to Example A (i.p.) and Example B (i.p.)). Also, oral (p.o.) administration of Example A (20 mg/kg/day) starting at 12 months of age and continued until the age of 15 months significantly reduces plaque formation as compared to the 15 month control (FIG. 1).

In an embodiment, Aβ plaque levels in brain as measured for a treatment group (FIG. 1A) or for individual subjects (FIG. 1B) is significantly reduced. Thus, in an embodiment, treatment with RAGE antagonist Example A at a dose of 10 mg/kg a day (i.p), or at a dose of 20 mg/kg a day (p.o), results in a reduction in plaque volume of 63%, and 47%, respectively. Also, treatment with RAGE antagonist Example B at a dose of 5 mg/kg/a day (i.p.) results in a reduction in plaque volume of 42%.

In an embodiment, RAGE antagonists not only able to stop the progression of Aβ deposition, but reverse the process. Thus, the plaque volume for mice treated with Example A (10/mg/kg/day i.p.) is actually lower than the starting (12 m) plaque volume (FIG. 1).

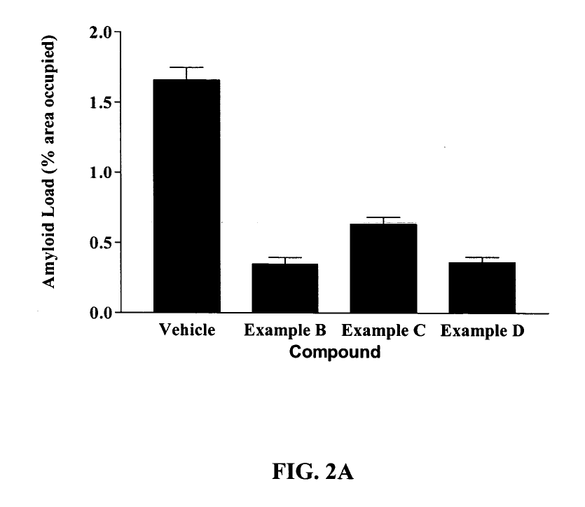

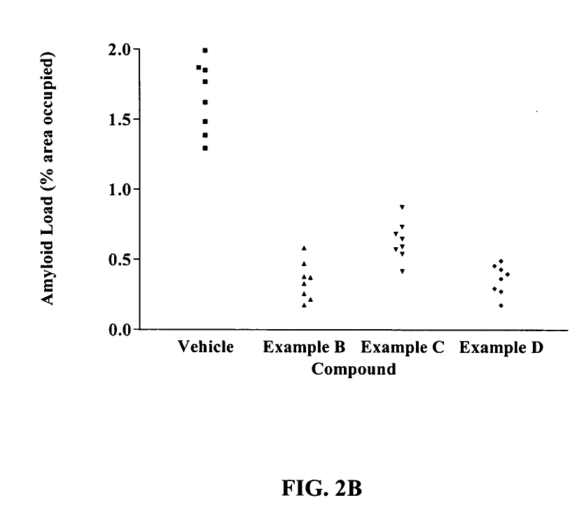

Also, in an embodiment, RAGE antagonists prevent the formation of Aβ plaques in the early-stages of AD. Thus, in an embodiment, treatment of 6 month old APP transgenic mice in the early stages of AD by injection of 5 mg/kg/day of RAGE antagonists Example B, Example C, and Example D, for 90 days (until 9 months) causes a significant reduction of Aβ amyloid in the brain (FIG. 2). The reduction is found across the treatment group (FIG. 2A) as well as for individual subjects (FIG. 2B).

RAGE Antagonists Improve Cognitive Function

In an embodiment, RAGE antagonists also reduce and reverse the behavioral effects seen with amyloid deposition. For example, treatment of 12 month old APP transgenic mice having established or later-stage AD with RAGE antagonists Example A and Example B for 3 months (until 15 months) reduces cognitive loss as compared to the vehicle control.

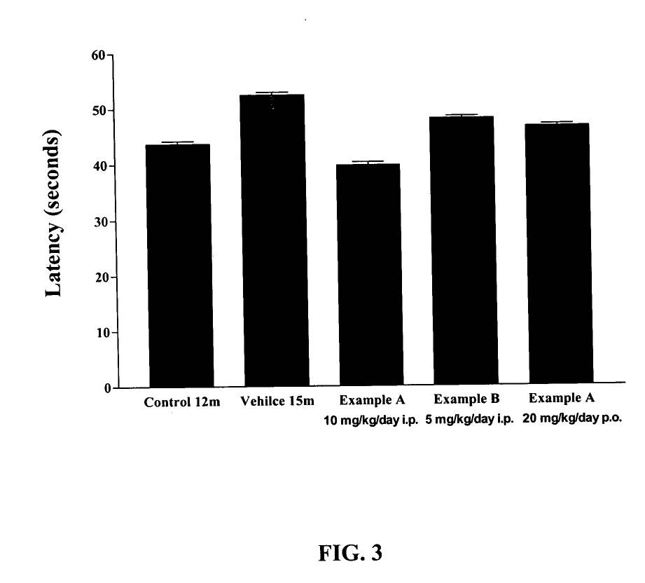

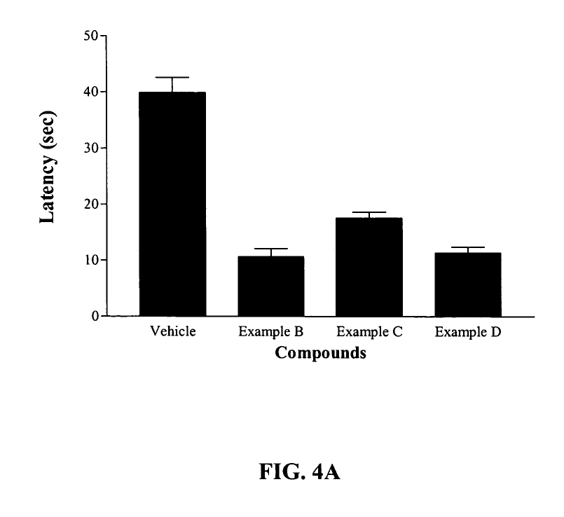

Thus, as shown in FIG. 3, treatment of 12 month old AD mice having with Example A at a dose of 10 mg/kg/day (i.p.) or 20 mg/kg/day (p.o.), or with Example B at a dose of 5 mg/kg/day (i.p.), improves cognition, measured as the time it takes the mice to find a hidden safety platform in a Morris water maze, compared to mice treated with vehicle only (15 m) (FIG. 3). In an embodiment, the RAGE antagonist Example A shows not only a decrease in latency time compared to the 15-month vehicle animals, but also as compared to the 12-month control animals, indicating that this treatment protocol not only prevents progress of Alzheimer's disease in the mice, but actually reverses the disease (FIG. 3).

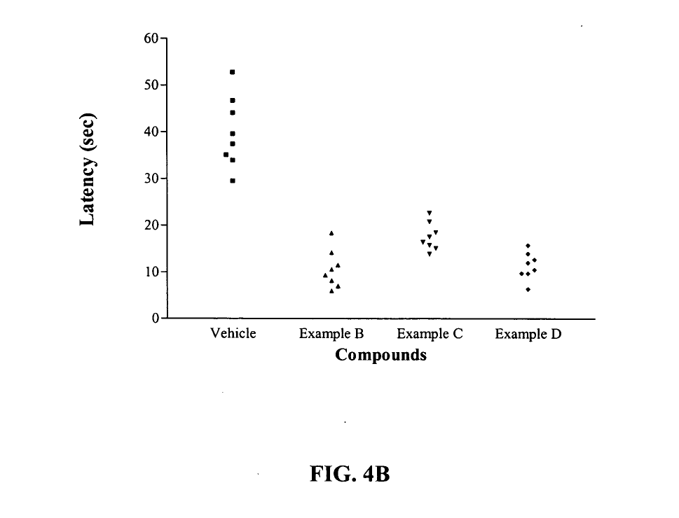

Also, in an embodiment, RAGE antagonists reduce cognitive loss in subjects in the early stages of AD. Thus, in an embodiment, treatment of 6 month old APP transgenic mice in the early stages of AD by injection of 5 mg/kg/day of RAGE antagonist Example B, Example C, and Example D, for 3 months (until 9 months), causes a reduction in latency time for learning (measured as the time it takes the mice to find a hidden safety platform in a water maze) compared to vehicle-treated controls. The reduction is found for treatment groups (FIG. 4A) and individual subjects (FIG. 4B).

Therapeutics

The invention further provides pharmaceutical compositions comprising the RAGE modulating compounds of the invention. In an embodiment, administration of RAGE antagonists may employ various routes as the antagonists are permeable across the blood-brain barrier. Thus, administration of the RAGE antagonists of the present invention may employ intraperitoneal injection. Alternatively, the RAGE antagonist may be administered orally, or as an aerosol. In another embodiment, administration of the compound is intravenous. In another embodiment, the RAGE antagonist is injected subcutaneously or adsorbed through the skin. For example, in an embodiment, the method of administration is by a transdermal patch. Also, administration may employ a time-release capsule. In another embodiment, administration of the compound is intra-arterial. In another embodiment, administration of the compound is sublingual. In yet another embodiment, administration of the drug is transrectal, as by a suppository or the like.

The term “pharmaceutical composition” is used herein to denote a composition that may be administered to a mammalian host, e.g., orally, parenterally, topically, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

The pharmaceutical compositions containing a compound of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, ydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alchol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as-COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxlate, maleate, pyruvate, malonate, succinate, citrate, tartarate, fumarate, mandelate, benzoate, cinnamate, methanesulfonate, ethanesulfonate, picrate and the like.

Other salts which are not pharmaceutically acceptable may be useful in the preparation of compounds of the invention and these form a further aspect of the invention.

Also provided by the present invention are prodrugs of the invention. In addition, some of the compounds of the present invention may form solvates with water or common organic solvents. Such solvates are also encompassed within the scope of the invention. Thus, in a further embodiment, there is provided a pharmaceutical composition comprising a compound of the present invention, or a pharmaceutically acceptable salt, solvate, or prodrug therof, and one or more pharmaceutically acceptable carriers, excipients, or diluents.

The compounds of the present invention may act as modulators of RAGE by binding to a single endogenous ligand to be advantageous in treatment of Alzheimer's disease and other disorders caused by amyloidosis, such as disorders characterized by excessive deposition of AL amyloid or AA amyloid plaques.

Further, the compounds of the present invention may act as modulators of RAGE interaction with two or more endogenous ligands in preference to others. Such compounds are advantageous in treatment of related or unrelated pathologies mediated by RAGE, such as Alzheimer's disease and other RAGE-mediated disorders.

Further, the compounds of the present invention may act as modulators of RAGE binding to each and every one of its ligands. In an embodiment, the compounds of the present invention prevent the downstream effect of RAGE, such as activation of NF-κB-regulated genes by cytokines IL-1 and TNF-α. Thus, antagonizing the binding of multiple physiological ligands to RAGE may prevent multiple pathophysiological consequences and is useful for management or treatment of AGE-RAGE interactions leading to Alzheimer's Disease and other RAGE-mediated disorders.

With respect to embodiments of the methods and compositions of the present invention, factors which will influence what constitutes an effective amount will depend upon the size and weight of the subject or individual being treated, the biodegradability of the therapeutic agent, the activity of the therapeutic agent, as well as its bioavailability. As used herein, the treated “subject” or “individual” includes mammalian subjects, preferably humans, who either suffer from one or more of the aforesaid diseases or disease states or are at risk for such. Accordingly, in the context of the therapeutic method of the invention, this method also includes treating a mammalian subject in a prophylactic manner, or prior to the onset of diagnosis such disease(s) or disease state(s).

In a further aspect of the present invention, the RAGE modulators of the invention are utilized in adjuvant therapeutic or combination therapeutic treatments with other known therapeutic agents.

The following is a non-exhaustive listing of adjuvants and additional therapeutic agents which may be utilized in combination with the RAGE modulators of the present invention:

Pharmacologic Classifications of Anticancer Agents for Use in AL Amyloidosis

1. Alkylating agents: Cyclophosphamide, nitrosoureas, carboplatin, cisplatin, procarbazine
2. Antibiotics: Bleomycin, Daunorubicin, Doxorubicin
3. Antimetabolites: Methotrexate, Cytarabine, Fluorouracil
4. Plant alkaloids: Vinblastine, Vincristine, Etoposide, Paclitaxel
5. Hormones: Tamoxifen, Octreotide acetate, Finasteride, Flutamide
6. Biologic response modifiers: Interferons, Interleukins
   Pharmacologic Classifications of Treatment for Rheumatoid Arthritis (Inflammation) for Use in AA Amyloidosis
1. Analgesics: Aspirin
2. NSAIDs (Nonsteroidal anti-inflammatory drugs): Ibuprofen, Naproxen, Diclofenac
3. DMARDs (Disease-Modifying Antirheumatic drugs): Methotrexate, gold preparations, hydroxychloroquine, sulfasalazine
4. Biologic Response Modifiers, DMARDs: Etanercept, Infliximab Glucocorticoids
   Pharmacologic Classifications of Treatment for Alzheimer's Disease and Other Disease of Aβ Amyloidosis
1. Cholinesterase Inhibitor: Tacrine, Donepezil
2. Antipsychotics: Haloperidol, Thioridazine
3. Antidepressants: Desipramine, Fluoxetine, Trazodone, Paroxetine
4. Anticonvulsants: Carbamazepine, Valproic acid

Thus, in a further preferred embodiment, the present invention provides methods and compositions for treating diseases of amyloidosis comprising administering to a subject in need thereof, a therapeutically effective amount of a compound of a RAGE antagonist in combination with a therapeutic agent comprising an alkylating agent, antimetabolite, plant alkaloid, antibiotic, hormone, biologic response modifier, analgesic, NSAID, DMARD, glucocorticoid, sulfonylurea, biguanide, insulin, cholinesterase inhibitor, antipsychotic, antidepressant, and anticonvulsant.

In an embodiment, the compositions comprising RAGE antagonists are administered at a dosage level of antagonist ranging from about 0.01 to 500 mg/kg/day, with alternate dosage ranges between 0.01 and 200 mg/kg/day, or between 0.1 to 100 mg/kg/day, or from 5 to 20 mg/kg/day. The amount of active ingredient that may be combined with the carrier materials to produce a single dosage will vary depending upon the host treated and the particular mode of administration. For example, a formulation intended for oral administration to humans may contain 1 mg to 2 grams of a RAGE antagonist such as Example A, Example B, Example C, or Example D, with an appropriate and convenient amount of carrier material which may vary from about 5 to 95 percent of the total composition. Dosage unit forms will generally contain between from about 5 mg to about 500 mg of active ingredient. The dosage can be individualized based on the specific clinical condition of the subject being treated. Thus, it will be understood that the specific dosage level for any particular subject will depend upon a variety of factors including the activity of the specific compound employed, the age, body weight, general health, sex, diet, time of administration, route of administration, rate of excretion, drug combination and the severity of the particular disease undergoing therapy.

### EXAMPLES

### Example 1

### Small Molecule RAGE Antagonists

RAGE antagonists of Formula (I) employed in Examples 2-6 are as follows.

| Reference | | |
|---|---|---|
| Name | Chemical Structure | Chemical Name |
| Example A | 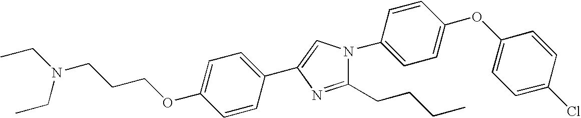 | [3-(4-{2-butyl-1-[4-(4- chloro-phenoxy)-phenyl]- 1H-imidazol-4-yl}- phenoxy)-propyl]-diethyl- amine |
| Example B | 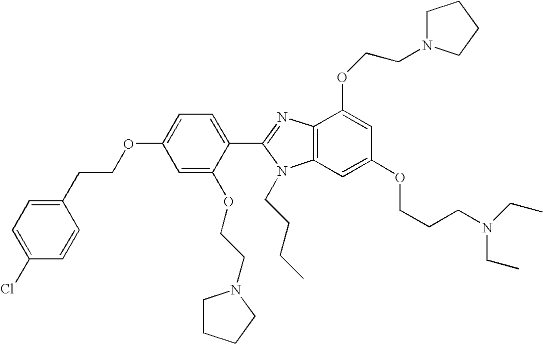 | {3-[3-butyl-2-[4-[2-(4- chloro-phenyl)-ethoxy]-2- (2-pyrrolidin-1-yl-ethoxy)- phenyl]-7-(2-pyrrolidin-1- yl-ethoxy)-3H- benzimidazol-5-yloxy]- propyl}-diethyl-amine |
| Example C | 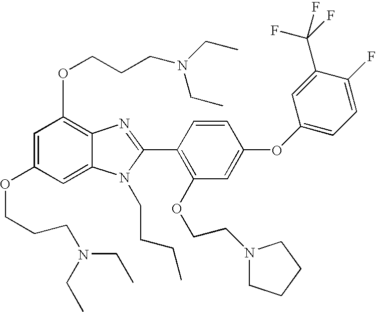 | (3-{1-Butyl-6-(3- diethylamino-propoxy)-2- [4-(4-fluoro-3- trifluoromethyl-phenoxy)- 2-(2-pyrrolidin-1-yl- ethoxy)-phenyl]-1H- benzoimidazol-4-yloxy}- propyl)-diethyl-amine |
| Example D | 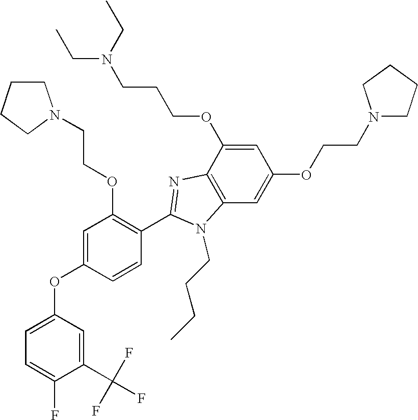 | {3-[1-Butyl-2-[4-(4-fluoro- 3-trifluoromethyl- phenoxy)-2-(2-pyrrolidin- 1-yl-ethoxy)-phenyl]-6-(2- pyrrolidin-1-yl-ethoxy)- 1H-benzoimidazol-4- yloxy]-propyl}-diethyl- amine |

### Example 2

### Methods and Materials

A. Study Design

For these experiments, the amyloid precursor protein (APP) transgenic model of mouse Aβ peptide amyloidosis was used. These animals begin to develop amyloid plaques at about 6 months age. APP transgenic mice were administered with vehicle or test compounds by intraperitoneal injection (i.p.) or orally (p.o.; per os), daily for 90 days. In studies of early AD, treatment started when the animals were 6 months old (25 g) (with plaques just beginning to form) and continued until the animals were 9 months old. In studies of established AD, treatment started at 12 months of age (35 g) and continued until the animals were 15 months old. At the end of the experiment, animals were sacrificed and examined for Aβ plaque burden in the brain (i.e., plaque volume).

B. In Vivo Methods

Male and female APP transgenic mice (Molecular Therapeutics, Inc.) of the appropriate age were given free access to food and water before and during the experiment. The animals were administered vehicle (saline) or the test compounds Example A (i.p. or p.o.) or Example B (i.p.), at doses which ranged from 5-20 mg/kg a day. Alternatively, test compounds Example B, Example C, and Example D were administered by intraperitoneal injection at 5 mg/kg/day. Test compounds were resuspended in saline to deliver 5-20 mg/kg based on the body weight of the animals.

C. Murine APP Transgenic Mice

The APP mice used in this experiment were generated by microinjection of the human APP gene into mouse eggs under the control of the platelet-derived growth factor B (PDGF-B) chain gene promoter (Games et al., Nature, 373:523-527 (1995)). The mice generated from this construct develop amyloid deposits starting at 6 months of age. In these experiments, animals were aged for either 6 or 12 months, and then maintained for 90 days under the selected experimental protocol and sacrificed for amyloid quantification.

D. Amyloid Load Determination

For histological examination, the animals were anesthetized by intraperitoneal injection of sodium pentobarbital (50 mg/kg). The animals were perfused transcardially with ice-cold phosphate-buffered saline (PBS) (4° C.) (10 mM NaPO₄, pH 7.2, 100 mM NaCl) followed by 4% paraformaldehyde. The brains were removed and placed in 4% paraformaldehyde over night. The brains were processed and embedded in paraffin. Ten serial 30-μm thick sections through the brain were obtained. Tissue sections were deparaffinized and washed in Tris buffered saline (TBS) pH 7.4 (10 mM Tris, pH 7.5; 100 mM NaCl) and blocked in the appropriate serum (mouse). Sections were blocked overnight at 4° C. and then incubated with 4G8 mouse monoclonal primary antibody which binds to Aβ peptide (Signet) overnight at 4° C. Sections were washed in TBS and secondary antibody was added and incubated for 1 hour at room temperature. After washing, the sections were incubated as instructed in the Vector ABC Elite kit and stained with diaminobenzoic acid (DAB). The reactions were stopped in water, treated with xylene and cover slips applied. The amyloid area in each section was determined using a computer-assisted image analysis system, consisting of a Power Macintosh computer equipped with a Quick Capture frame grabber card, a Hitachi CCD camera mounted on an Olympus microscope, a camera stand, and NIH Image Analysis Software (v. 1.55). The images were captured and the total area of amyloid was determined over ten sections. A single operator blinded to treatment status performed all measurements. Summing the amyloid volumes of the sections (measured as the percent amyloid for the section) and dividing by the total number of sections was used to calculate the percent amyloid volume in the brain.

E. Behavioral Analysis

Water-maze testing was used as a measure of cognitive function. Mice were trained in a 1.2 meter open field water maze. The pool was filled to a depth of 20 cm with water and maintained at 25° C. An escape platform (10 cm in diameter) was placed 1 cm below the surface of the water. During the trials, the platform was removed from the pool.

For the cued training sessions, the platform was marked with a 10 cm×1 cm stick painted black. The cued test was carried out in the pool surrounded with white curtains to hide any extra-maze cues. All animals underwent non-spatial pretraining (NSP) for three consecutive days. For the training and learning studies, the curtains were removed to extra maze cues (this allowed for identification of animals with swimming impairments).

Initially, the mice were placed on the hidden platform for 20 seconds (trial 1). For trials 2 and 3, animals were released in the water at a distance of 10 cm from the cued-platform or hidden platform (trial 4) and allowed to swim to the platform. On the second day of trails, the hidden platform was moved randomly between the center of the pool or the center of each quadrant. The animal was released into the pool, randomly facing the wall and was allowed 90 seconds to reach the platform. On the third day, animals were given three trials, two with a hidden platform and one with a cued platform.

Two days following the NSP, animals were subjected to behavioral trials. For these trials, the platform was placed in the center of one quadrant of the pool and the animals released facing the wall in a random fashion. The animal was allowed to find the platform or swim for 60 seconds.

F. Statistical Analysis

The results are expressed as the mean±standard deviation (SD). Significance was analyzed using a t-test.

G. Exclusion of Animals from the Study

Animals were to be excluded from the study based upon several criteria:
1. Animals that died prior to completion of study (at any point).
2. Animals that developed severe complications following administration of compounds.

H. Treatment Groups

All groups were subjected to the experimental compounds or were controls. For the study of established AD, a total of 38 animals each 12 months old were subjected to administration of vehicle or test compounds (Table 1). In addition, a fifth group of animals was assessed at 12 month as the zero time-point since this is the time-point at which these mice begin to show significant development of amyloid plaques. These animals provided the starting point control.

TABLE 1
APP Mouse Model of 12 month old AD
animals treated for 3 months

| Group | Compound | Dose (mg/kg/day) | Route |
|---|---|---|---|
| 1 (n = 8 mice) | Vehicle (saline) | — | IP |
| 2 (n = 8 mice) | Example A | 10 | IP |
| 3 (n = 8 mice) | Example B | 5 | IP |
| 4 (n = 8 mice) | Example A | 20 | PO |
| 5 (n = 6 mice) | control | — | — |

For the study of early AD, 8 mice (each 6 months of age) were treated with the vehicle and 32 mice (each 6 months of age) (8 mice per treatment group) were injected daily (i.p., for 3 months) with 5 mg/kg per day of Example B, Example C, or Example D.

### Example 3

### Effect of RAGE Antagonists on Aβ Amyloidosis in Mice with Established AD

The amyloid load per mouse was determined from APP transgenic mice. Data from mice with Aβ amyloid that were administered vehicle, or RAGE antagonists Example A or Example B were examined.

In this mouse model, AD begins to develop by about 6-12 months. Intraperitoneal (i.p.) injection of Example A at a dose of 10 mg/kg per day, or of Example B at a dose of 5 mg/kg/day into 12 month old APP transgenic mice having established AD for 3 months (i.e., until 15 months) reduced plaque formation as compared to age-matched AD mice injected with saline (Table 2). FIG. 1 shows the reduction in plaque for AD mice injected with either Example A or Example B as compared to age-matched AD mice injected with saline (15 m control) (FIG. 1: compare month (m) control to Example A (i.p.) and Example B (i.p.)). Also, oral (p.o.) administration of Example A (20 mg/kg/day) starting at 12 months of age and continued for 3 months until the age of 15 months significantly reduced plaque formation as compared to the 15 month control.

Thus, compared with the vehicle-injected group, the brain amyloid load was significantly decreased in all of the groups treated with the RAGE antagonists. It was found that Aβ peptide levels in brain as measured for the group (FIG. 1A) or for individual animals (FIG. 1B) was significantly reduced. Thus, mice treated with Example A at a dose of 10 mg/kg a day (i.p), or at a dose of 20 mg/kg a day (p.o), showed a reduction in plaque volume of 63%, and 47%, respectively. Also, mice treated with Example B at a dose of 5 mg/kg/a day (i.p.) showed a reduction in plaque volume of 42% (Table 2).

Example A (i.p.) showed a larger change in the decrease in amyloid load when compared to the other treatments. Interestingly, Example A (i.p.) also demonstrated an appreciable reversal in amyloid load compared to the 12 month time point, indicating that this treatment actually reduces the volume of pre-existing plaques. Thus, the plaque volume for mice treated with Example A (i.p.) was actually lower than the starting plaque volume measured as animals at the 12 month timepoint of the disease (FIG. 1). There were no deaths in the study.

TABLE 2
Percent decrease in amyloid in the brain upon
treatment with RAGE antagonists

| | Percent reduction in Aβ |
|---|---|
| Compound | amyloid* |
| Vehicle | 0% |
| Example A (i.p., 10 mg/kg/day) | 63% |
| Example B (i.p., 5 mg/kg/day) | 42% |
| Example A (p.o., 20 mg/kg/day) | 47% |

*Percent decreases are compared to the 15 m vehicle control animals.

### Example 4

### Effect of RAGE Antagonists on Aβ Amyloidosis in Mice with Early AD

In these experiments, the amyloid load per mouse was determined for 9 month old APP transgenic mice with early AD. In this experiment, APP transgenic mice were injected (i.p.) for 3 months (beginning at 6 months of age) with saline vehicle, or 5/mg/kg per day RAGE antagonist compounds (Example B, Example C, or Example D) in saline.

Compared with the vehicle-injected group, the amyloid load in the brains was significantly decreased in all of the treated groups to varying degrees (FIG. 2 and Table 3). There were no deaths in this study.

TABLE 3
Percent decrease in amyloid in the
brain (i.p. injections at 5 mg/kg/day)

| | Percent reduction in Aβ amyloid |
|---|---|
| Compound | (5 mg/kg/day) |
| Vehicle | 0% |
| Example B | 79% |
| Example C | 62% |
| Example D | 78% |

Percent decreases are compared to the vehicle control animals

### EXAMPLE 5

### Effect of Administration of Compounds on Behavioral Measures in Mice with Established AD

The behavioral effects of treatment with Example A and Example B were determined in the 15 month old APP transgenic mice (mice having established AD at the zero time-point of 12 months). At the termination of the treatment protocol (comprising injection of 12 month old AAP transgenic mice for 3 months with saline, Example A, or Example B as described above), mice were subjected to the Morris water maze task (Morris et al., Nature, 297:681-683 (1982)) and the latency period for the mice to find a hidden platform was determined. As shown in Table 4, treatment with the RAGE antagonist compounds Example A and Example B (either i.p. or p.o.) reduced the latency period for the mice to find the platform as compared to the 15 month vehicle control. Thus, intraperitoneal (i.p.) injection of Example A at a dose of 10 mg/kg per day, or of Example B at a dose of 5 mg/kg/day, starting at 12 months of age until the age of 15 months, improved cognition in mice with established AD (FIG. 3: compare 15 month (15 m) control to Example A (i.p.) and Example B (i.p.)). Also, oral (p.o.) administration of Example A (20 mg/kg/day), starting at 12 months of age until the age of 15 months significantly reduced the latency period for the mice to find the platform as compared to the 15 month control (FIG. 3).

Thus, mice treated with Example A at a dose of 10 mg/kg a day (i.p), or at a dose of 20 mg/kg a day (p.o) showed a reduction in latency time for learning of 24%, and 11%, respectively (Table 4). Also, mice treated with Example B at a dose of 5 mg/kg/a day (i.p.) showed a reduction in latency time for learning of 8% (Table 4). These changes were found to be statistically significant (p<0.0001 for all test article groups as compared to the vehicle control).

Also, the RAGE antagonist Example A, when administered at a dose of 10 mg/kg/day (i.p.), not only a decreased latency time compared to the 15-month controls, but also as compared to the 12-month control animals (FIG. 3). Thus, Example A improved cognitive function to levels better than the levels at the starting timepoint (12 months), indicating that in mice with established AD, RAGE antagonist Example A not only reduced amyloid deposition, but at least partially reversed the cognitive loss associated with amyloid deposition.

Overall, the results suggest that RAGE antagonists slow the process of amyloid formation and can reverse the process of amyloid deposition and associated behavioral deficits.

TABLE 4
Behavioral latency period in APP transgenic
mice treated with compounds

| Compound | Latency Period* |
|---|---|
| Vehicle | 0% |
| Example A (i.p., 10 mg/kg/day) | 24% |
| Example B (i.p., 5 mg/kg/day) | 8% |
| Example A (p.o., 20 mg/kg/day) | 11% |

Percent reduction in latency compared to the 15 m vehicle control animals

### EXAMPLE 6

### Effect of Administration of Compounds on Behavioral Measures in Mice with Early AD

The behavioral effects of treatment with the RAGE antagonists, Example B, Example C, and Example D, were determined using the 9 month old APP transgenic mice having early AD. At the termination of the treatment protocol (comprising injection of 6 month old APP transgenic mice for 3 months with a saline vehicle or 5 mg/kg/day of a RAGE antagonists Example B, Example C, and Example D, in saline), mice were subjected to the Morris water maze task (Morris et al., Nature, 297:681-683 (1982)) and the latency period for the mice to find a hidden platform was determined. As shown in Table 5, treatment with the RAGE antagonists, Example B, Example C, and Example D, reduced the latency period for the mice to find the platform compared to the vehicle control. This shows that RAGE antagonists not only protect against amyloid formation, but also protect the animal from cognitive loss associated with amyloid deposition.

TABLE 5
Behavioral latency period in APP transgenic
mice treated with compounds

| Compound | Latency Period* |
|---|---|
| Vehicle | 0% |
| Example B | 73% |
| Example C | 56% |
| Example D | 72% |

*Percent change compared to the vehicle control animals

Thus, this study shows that small organic molecule RAGE antagonists, when administered to mice in the early stages of AD (6 month old APP transgenic mice), or to mice in the later stages of AD (12 month old APP transgenic mice), can reduce and even reverse amyloidosis and the behavioral deficits associated with amyloid plaque formation. These data show that RAGE antagonists are viable candidates for the treatment of amyloid diseases.

While the invention has been described and illustrated with reference to certain preferred embodiments thereof, those skilled in the art will appreciate that various changes, modifications and substitutions can be made therein without departing from the spirit and scope of the invention. For example, effective dosages other than the preferred dosages as set forth herein may be applicable as a consequence of variations in the responsiveness of the mammal being treated for conditions and diseases of amyloidosis. Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

1. A composition to reverse pre-existing amyloidosis in an individual in need thereof comprising a pharmacologically effective amount of a RAGE antagonist in a pharmaceutically acceptable carrier, wherein a pharmacologically effective amount of antagonist comprises sufficient RAGE antagonist to reduce pre-existing amyloid plaques in the individual.

2. The composition of claim 1, wherein a pharmacologically effective amount of the RAGE antagonist reverses symptoms associated with amyloidosis.

3. The composition of claim 1, wherein the individual is suffering from a disease of abnormal amyloid accumulation.

4. The composition of claim 1, wherein the amyloid plaque reduced by the RAGE antagonist comprises an amyloid-β (Aβ) plaque.

5. The composition of claim 1, wherein the plaque reduction occurs, at least in part, in the individual's brain.

6. The composition of claim 1, wherein the amyloidosis causes Alzheimer's Disease (AD) and the reversal of symptoms associated with amyloidosis is associated with improved cognition.

7. The composition of claim 1, wherein the amyloidosis is associated with systemic amyloid deposition.

8. The composition of claim 7, wherein the amyloidosis comprises amyloid-light chain amyloidosis (AL amyloidosis) or amyloid-associated amyloidosis (AA amyloidosis).

9. The composition of claim 1, wherein the RAGE antagonist comprises an organic compound having a molecular weight less than 1000 Da.

10. The composition of claim 9, wherein the RAGE antagonist comprises compounds of Formula (I) 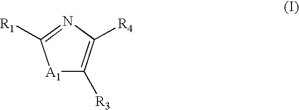
wherein for the compounds of Formula 1:
R₁ comprises -hydrogen, -aryl, -heteroaryl, -cycloalkyl, -heterocyclyl, -alkyl, -alkenyl, -alkynyl, -alkylene-aryl, -alkylene-heteroaryl, -alkylene-heterocyclyl, -alkylene-cycloalkyl, -fused cycloalkylaryl, -fused cycloalkylheteroaryl, -fused heterocyclylaryl, -fused heterocyclylheteroaryl, -alkylene-fused cycloalkylaryl, -alkylene-fused cycloalkylheteroaryl, -alkylene-fused heterocyclylaryl, -alkylene-fused heterocyclylheteroaryl, or -G₁-G₂-G₃—R₅,
wherein
G₁ and G₃ independently comprise alkylene, alkenylene, alkynylene, cycloalkylene, heterocyclylene, arylene, heteroarylene, (aryl)alkylene, (heteroaryl) alkylene, (aryl)alkenylene, (heteroaryl)alkenylene, or a direct bond;
G₂ comprises —O—, —S—, —S(O)—, —N(R₆)—, —S(O)₂—, —C(O)—, —O—C(O)—, —C(O)—O—, —C(O)N(R₆)—, —N(R₆)C(O)—, —S(O₂)N(R₆)—, N(R₆)S(O₂)—, —O-alkylene-C(O)—, —(O)C-alkylene-O—, —O-alkylene-, -alkylene-O—, alkylene, alkenylene, alkynylene, cycloalkylene, heterocyclylene, arylene, heteroarylene, fused cycloalkylarylene, fused cycloalkylheteroarylene, fused heterocyclylarylene, fused heterocyclylheteroarylene, or a direct bond, wherein R₆ comprises hydrogen, aryl, alkyl, -alkylene-aryl, alkoxy, or -alkylene-O-aryl; and
R₅ comprises hydrogen, aryl, heteroaryl, cycloalkyl, heterocyclyl, alkyl, alkenyl, alkynyl, -alkylene-aryl, -alkylene-heteroaryl, -alkylene-heterocyclyl, -alkylene-cycloalkyl, fused cycloalkylaryl, fused cycloalkylheteroaryl, fused heterocyclylaryl, fused heterocyclylheteroaryl, -alkylene-fused cycloalkylaryl, -alkylene-fused cycloalkylheteroaryl, -alkylene-fused heterocyclylaryl, or -alkylene-fused heterocyclylheteroaryl;
A₁ comprises O, S, or —N(R₂)—;
wherein R₂ comprises
a) —H;
b) -aryl;
c) -heteroaryl;
d) -cycloalkyl
e) heterocyclyl;
f) -alkyl;
g) -alkenyl;
h) -alkynyl;
i) -alkylene-aryl,
j) -alkylene-heteroaryl,
k) -alkylene-heterocyclyl,
l) -alkylene-cycloalkyl;
m) -fused cycloalkylaryl,
n) -fused cycloalkylheteroaryl,
o) -fused heterocyclylaryl,
p) -fused heterocyclylheteroaryl;
q) -alkylene-fused cycloalkylaryl,
r) -alkylene-fused cycloalkylheteroaryl,
s) -alkylene-fused heterocyclylaryl,
t) -alkylene-fused heterocyclylheteroaryl; or
u) a group of the formula 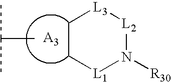
wherein
A₃ comprises an aryl or heteroaryl group;
L₁ and L₂ independently comprise alkylene or alkenylene; and
L₃ comprises a direct bond, alkylene, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 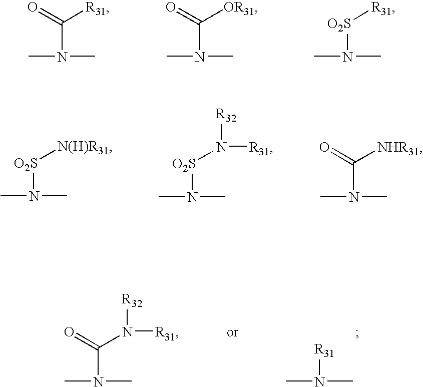
wherein R₃₀, R₃₁, and R₃₂ independently comprise hydrogen, aryl, heteroaryl, alkyl, alkylene-aryl, or -alkylene-heteroaryl;
R₃ and R₄ independently comprise
a) -hydrogen,
b) -halogen,
c) -hydroxyl,
d) -cyano,
e) -carbamoyl,
f) -carboxyl,
g) -aryl,
h) -heteroaryl,
i) -cycloalkyl,
j) -heterocyclyl,
k) -alkyl,
l) -alkenyl,
m) -alkynyl,
n) -alkylene-aryl,
o) -alkylene-heteroaryl,
p) -alkylene-heterocyclyl,
q) -alkylene-cycloalkyl,
r) -fused cycloalkylaryl,
s) -fused cycloalkylheteroaryl,
t) -fused heterocyclylaryl,
u) -fused heterocyclylheteroaryl,
v) -alkylene-fused cycloalkylaryl,
w) -alkylene-fused cycloalkylheteroaryl,
x) -alkylene-fused heterocyclylaryl,
y) -alkylene-fused heterocyclylheteroaryl;
z) —C(O)—O-alkyl;
aa) —C(O)—O-alkylene-aryl;
bb) —C(O)—NH-alkyl;
cc) —C(O)—NH-alkylene-aryl;
dd) —SO₂-alkyl;
ee) —SO₂-alkylene-aryl;
ff) —SO₂-aryl;
gg) —SO₂—NH-alkyl;
hh) —SO₂—NH— alkylene-aryl;
ii) —C(O)-alkyl;
jj) —C(O)-alkylene-aryl;
kk) —G₄-G₅-G₆—R₇;
ll) —Y₁-alkyl;
mm) —Y₁-aryl;
nn) —Y₁-heteroaryl;
oo) —Y₁-alkylene-aryl;
pp) —Y₁-alkylene-heteroaryl;
qq) —Y₁-alkylene-NR₉R₁₀; or
rr) —Y₁-alkylene-W₁—R₁₁;
wherein
G₄ and G₆ independently comprise alkylene, alkenylene, alkynylene, cycloalkylene, heterocyclylene, arylene, heteroarylene, (aryl)alkylene, (heteroaryl)alkylene, (aryl)alkenylene, (heteroaryl)alkenylene, or a direct bond;
G₅ comprises —O—, —S—, —N(R₈)—, —S(O)—, —S(O)₂—, —C(O)—, —O—C(O)—, —C(O)—O—, —C(O)N(R₈)—, N(R₉)C(O)—, —S(O₂)N(R₈)—, N(R₈)S(O₂)—, —O-alkylene-C(O), —(O)C-alkylene-O—, —O-alkylene-, -alkylene-O—, alkylene, alkenylene, alkynylene, cycloalkylene, heterocyclylene, arylene, heteroarylene, fused cycloalkylarylene, fused cycloalkylheteroarylene, fused heterocyclylarylene, fused heterocyclylheteroarylene, or a direct bond, wherein R₈ comprises -hydrogen, -aryl, -alkyl, -alkylene-aryl, or -alkylene-O-aryl;
R₇ comprises hydrogen, aryl, heteroaryl, cycloalkyl, heterocyclyl, alkyl, alkenyl, alkynyl, alkylene-aryl, -alkylene-heteroaryl, -alkylene-heterocyclyl, -alkylene-cycloalkyl, fused cycloalkylaryl, fused cycloalkylheteroaryl, fused heterocyclylaryl, fused heterocyclylheteroaryl, alkylene-fused cycloalkylaryl, -alkylene-fused cycloalkylheteroaryl, -alkylene-fused heterocyclylaryl, or -alkylene-fused heterocyclylheteroaryl;
Y₁ and W₁ independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—, 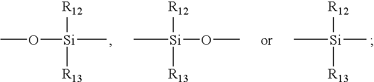
wherein R₁₂ and R₁₃ independently comprise aryl, alkyl, -alkylene-aryl, alkoxy, or -alkylene-O-aryl; and
R₉, R₁₀, and R₁₁ independently comprise aryl, heteroaryl, alkyl, -alkylene-heteroaryl, or -alkylene-aryl; and R₉ and R₁₀ may be taken together to form a ring having the formula —(CH₂)ₒ—X₁—(CH₂)ₚ— bonded to the nitrogen atom to which R₉ and R₁₀ are attached,
wherein
o and p are, independently, 1, 2, 3, or 4; and
X₁ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 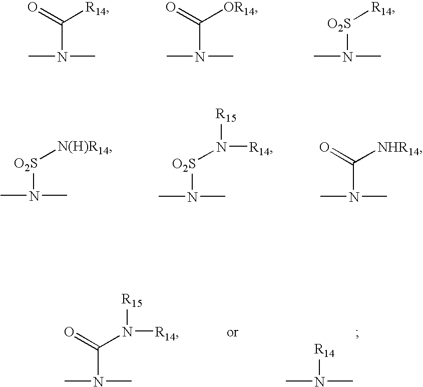
wherein R₁₄ and R₁₅ independently hydrogen, aryl, heteroaryl, alkyl, -alkylene-aryl, or -alkylene-heteroaryl;
wherein
the aryl and/or alkyl group(s) in R₁, R₂, R₃, R₄, R₅, R₆, R₇, R₈, R₉, R₁₀, R₁₁, R₁₂, R₁₃, R₁₄, and R₁₅ may be optionally substituted 1-4 times with a substituent group, wherein said substituent group(s) or the term substituted refers to a group comprising:
a) —H,
b) -halogen,
c) -hydroxyl,
d) -cyano,
e) -carbamoyl,
f) -carboxyl,
g) —Y₂-alkyl;
h) —Y₂-aryl;
i) —Y₂-heteroaryl;
j) —Y₂— alkylene-heteroarylaryl;
k) —Y₂-alkylene-aryl;
l) —Y₂-alkylene-W₂—R₁₈;
q) —Y₃—Y₄—NR₂₃R₂₄,
r) —Y₃—Y₄—NH—C(═NR₂₅)NR₂₃R₂₄,
s) —Y₃—Y₄ (═NR₂₅)NR₂₃R₂₄, or
t) —Y₃—Y₄—Y₅-A₂,
wherein
Y₂ and W₂ independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—, 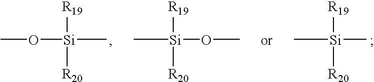
wherein;
R₁₉ and R₂₀ independently comprise hydrogen, aryl, alkyl, -alkylene-aryl, alkoxy, or -alkylene-O-aryl; and
R₁₈ comprises aryl, alkyl, -alkylene-aryl, -alkylene-heteroaryl, and -alkylene-O-aryl;
Y₃ and Y₅ independently comprise a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—, 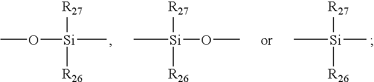
wherein R₂₇ and R₂₆ independently comprise aryl, alkyl, -alkylene-aryl, alkoxy, or -alkyl-O-aryl;
Y₄ comprises
a) -alkylene;
b) -alkenylene;
c) -alkynylene;
d) -arylene;
e) -heteroarylene;
f) -cycloalkylene;
g) -heterocyclylene;
h) -alkylene-arylene;
i) -alkylene-heteroarylene;
j) -alkylene-cycloalkylene;
k) -alkylene-heterocyclylene;
l) -arylene-alkylene;
m) -heteroarylene-alkylene;
n) -cycloalkylene-alkylene;
o) -heterocyclylene-alkylene;
p) —O—;
q) —S—;
r) —S(O₂)—; or
s) —S(O)—;
wherein said alkylene groups may optionally contain one or more O, S, S(O), or SO₂ atoms;
A₂ comprises
a) heterocyclyl, fused arylheterocyclyl, or fused heteroarylheterocyclyl, containing at least one basic nitrogen atom,
b) -imidazolyl, or
c) -pyridyl; and
R₂₃, R₂₄, and R₂₅ independently comprise hydrogen, aryl, heteroaryl, -alkylene-heteroaryl, alkyl, -alkylene-aryl, -alkylene-O-aryl, or -alkylene-O-heteroaryl; and R₂₃ and R₂₄ may be taken together to form a ring having the formula —(CH₂)ₙ—X₃—(CH₂)ₜ— bonded to the nitrogen atom to which R₂₃ and R₂₄ are attached
wherein
s and t are, independently, 1, 2, 3, or 4;
X₃ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 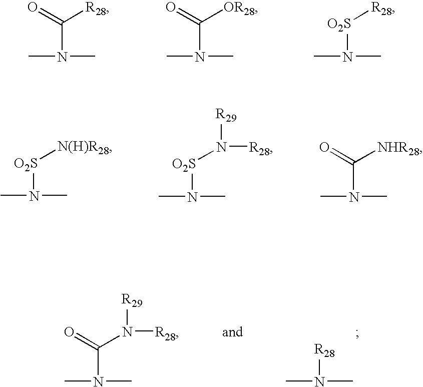
wherein R₂₈ and R₂₉ independently comprise hydrogen, aryl, heteroaryl, alkyl, -alkylene-aryl, or -alkylene-heteroaryl;
wherein
either
at least one of the groups R₁, R₂, R₃ and R₄ are substituted with at least one group of the formula —Y₃—Y₄—NR₂₃R₂₄, —Y₃—Y₄—NH—C(═NR₂₅)NR₂₃R₂₄, —Y₃—Y₄-C(═NR₂₅)NR₂₃R₂₄, or —Y₃—Y₄—Y₅-A₂, with the proviso that no more than one of R₂₃, R₂₄, and R₂₅ may comprise aryl or heteroaryl; or
R₂ is a group of the formula 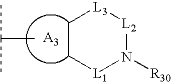
and
wherein
one of R₃ and R₄, R₃ and R₂, or R₁ and R₂, may be taken together to constitute, together with the atoms to which they are bonded, an aryl, heteroaryl, fused arylcycloalkyl, fused arylheterocyclyl, fused heteroarylcycloalkyl, or fused heteroarylheterocyclyl ring system,
wherein
said ring system or R₁, R₂, R₃, or R₄ is substituted with at least one group of the formula
a) —Y₅—Y₆—NR₃₃R₃₄;
b) —Y₅—Y₆—NH—C(═NR₃₅)NR₃₃R₃₄;
c) —Y₅—Y₆—C(═NR₃₅)NR₃₃R₃₄; or
d) —Y₅—Y₆—Y₇-A₄;
wherein
Y₅ and Y₇ independently comprise a direct bond, —CH₂—, —O—, —N(H), —S—, SO₂—, —CON(H)—, —NHC(O)—, —NHCON(H)—, —NHSO₂—, —SO₂N(H)—, C(O)—O—, —NHSO₂NH—, —O—CO—, 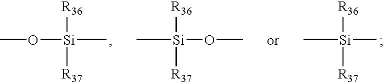
wherein R₃₆ and R₃₇ independently comprise aryl, alkyl, -alkylene-aryl, alkoxy, or -alkyl-O-aryl;
Y₆ comprises
a) alkylene;
b) alkenylene;
c) alkynylene;
d) arylene;
e) heteroarylene;
f) cycloalkylene;
g) heterocyclylene;
h) alkylene-arylene;
i) alkylene-heteroarylene;
j) alkylene-cycloalkylene;
k) alkylene-heterocyclylene;
l) arylene-alkylene;
m) heteroarylene-alkylene;
n) cycloalkylene-alkylene;
o) heterocyclylene-alkylene;
p) —O—;
q) —S—;
r) —S(O₂)—; or
s) —S(O)—;
wherein said alkylene groups may optionally contain one or more O, S, S(O), or SO₂ atoms;
A₄ comprises
a) heterocyclyl, fused arylheterocyclyl, or fused heteroarylheterocyclyl, containing at least one basic nitrogen atom,
b) -imidazolyl, or
c) -pyridyl; and
R₃₃, R₃₄ and R₃₅ independently comprise hydrogen, aryl, heteroaryl, alkyl, -alkylene-aryl, or -alkylene-O-aryl; with the proviso that no two of R₃₃, R₃₄ and R₃₅ are aryl and/or heteroaryl; and R₃₃ and R₃₄ may be taken together to form a ring having the formula —(CH₂)ₙ—X₄—(CH₂)ᵥ— bonded to the nitrogen atom to which R₃₃ and R₃₄ are attached, wherein
u and v are, independently, 1, 2, 3, or 4;
X₄ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 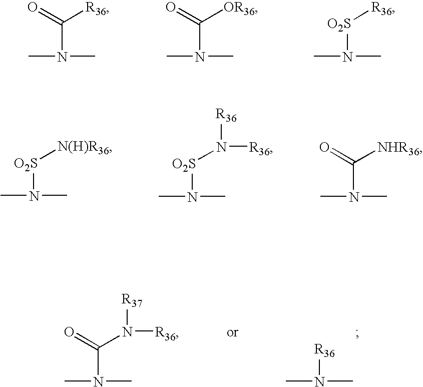
wherein R₃₆ and R₃₇ independently comprise hydrogen, aryl, heteroaryl, alkyl, -alkylene-aryl, or -alkylene-heteroaryl; and
wherein said ring system is optionally substituted with substituents comprising
a) —H;
b) -halogen;
c) -hydroxyl;
d) -cyano;
e) -carbamoyl;
f) -carboxyl;
g) —Y₈-alkyl;
h) —Y₈-aryl;
i) —Y₈-heteroaryl;
j) —Y₈-alkylene-aryl;
k) —Y₈-alkylene-heteroaryl;
l) —Y₈-alkylene-NR₃₈R₃₉; or
m) —Y₈-alkylene-W₃—R₄₀;
wherein
Y₈ and W₃ independently comprise —CH₂—, —O—, —N(H), —S—, SO₂—, —CON(H)—, —NHC(O)—, —NHCON(H)—, —NHSO₂—, —SO₂N(H)—, C(O)—O—, —NHSO₂NH—, —O—CO—, 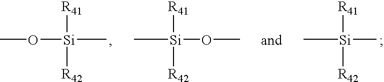
wherein R₄₁ and R₄₂ independently comprise aryl, alkyl, -alkylene-aryl, alkoxy, or -alkyl-O-aryl; and
R₃₈, R₃₉, and R₄₀ independently comprise hydrogen, aryl, alkyl, -alkylene-aryl, -alkylene-heteroaryl, and -alkyene-O-aryl; and R₃₈ and R₃₉ may be taken together to form a ring having the formula —(CH₂)_{w}—X₇—(CH₂)ₙ— bonded to the nitrogen atom to which R₃₈ and R₃₉ are attached wherein
w and x are, independently, 1, 2, 3, or 4;
X₇ comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 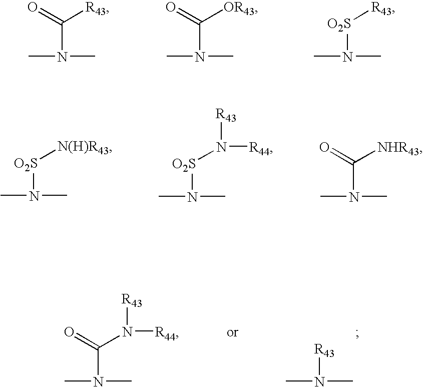
wherein R₄₃ and R₄₄ independently comprise hydrogen, aryl, heteroaryl, alkyl, -alkylene-aryl, or -alkylene-heteroaryl;
or a pharmaceutically acceptable salt thereof.

11. The composition of claim 10, wherein the RAGE antagonist comprises

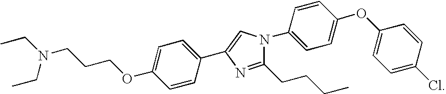

12. The composition of claim 10, wherein the RAGE antagonist comprises

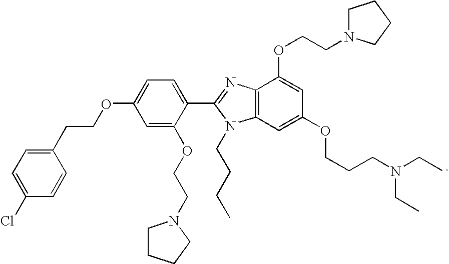

13. The composition of claim 10, wherein the RAGE antagonist comprises

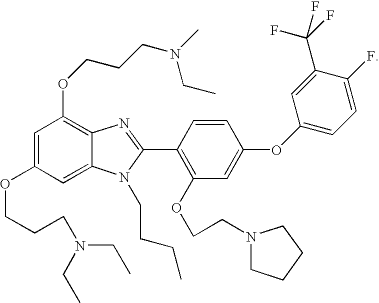

14. The composition of claim 10, wherein the RAGE antagonist comprises

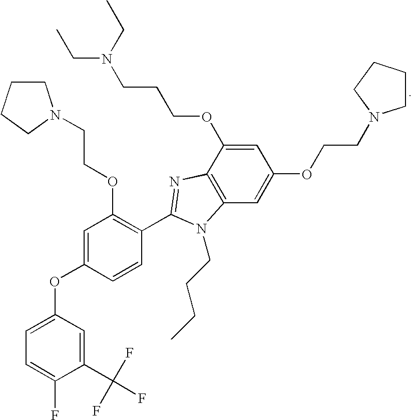

15. The composition of claim 9, wherein the RAGE antagonist comprises compounds of Formula (II) 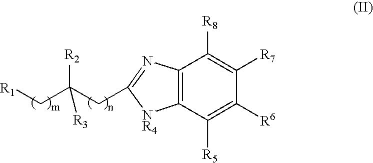
wherein for compounds of Formula (II)
m is an integer of from 0 to 3;
n is an integer of from 0 to 3;
R₁ comprises aryl;
R₂ comprises
a) a group of the formula —N(R₉R₁₀), —NHC(O)R₉, or —NHC(O)OR₉;
b) a group of the formula —OR₉;
c) a group of the formula —SR₉, —SOR₉, —SO₂R₉, —SO₂NHR₉, or —SO₂N(RgR₁₀);
wherein R₉ and R₁₀ independently comprise
1) —H;
2) -Aryl;
3) a group comprising
a) -C₁₋₆ alkyl;
b) —C₁₋₆ alkylaryl;
c) 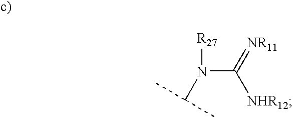
d) -aryl;
e) —C₁₋₆ alkyl; or
f) —C₁₋₆ alkylaryl;
R₃ and R₄ independently comprise
a) H;
b) -aryl;
c) C₁₋₆ alkyl;
d) —C₁₋₆ alkylaryl; or
e) —C₁₋₆ alkoxyaryl;
R₅, R₆, R₇, and R₈ independently comprise
a) —H;
b) —C₁₋₆ alkyl;
c) -aryl;
d) —C₁₋₆ alkylaryl;
e) —C(O)—O—C₁₋₆ alkyl;
f) —C(O)—O—C₁₋₆ alkylaryl;
g) —C(O)—NH—C₁₋₆ alkyl;
h) —C(O)—NH—C₁₋₆ alkylaryl;
i) —SO₂—C₁₋₆ alkyl;
j) —SO₂—C₁₋₆ alkylaryl;
k) —SO₂-aryl;
l) —SO₂—NH—C₁₋₆ alkyl;
m) —SO₂—NH—C₁₋₆ alkylaryl;
n) —C(O)—C₁₋₆ alkyl;
o) —C(O)—C₁₋₆ alkylaryl;
p) —Y—C₁₋₆ alkyl;
q) —Y-aryl;
r) —Y—C₁₋₆ alkylaryl;
s) —Y—C₁₋₆ alkylene-NR₁₃R₁₄; or
t) —Y—C₁₋₆alkylene-W—R₁₅;
wherein Y and W independently comprise —CH₂—, —O—, —N(H)—, —S—, SO₂—, —CON(H)—, —NHC(O)—, —NHCON(H)—, —NHSO₂—, —SO₂N(H)—, —C(O)—O—, —NHSO₂NH—, —O—CO—, 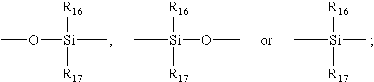
R₁₆ and R₁₇ independently comprise aryl, C₁-C₆ alkyl, C₁-C₆ alkylaryl, C₁-C₆ alkoxy, or C₁-C₆ alkoxyaryl;
R₁₅ independently comprise aryl, C₁-C₆ alkyl, or C₁-C₆ alkylaryl; or
u) halogen, hydroxyl, cyano, carbamoyl, or carboxyl;
R₁₁, R₁₂, R₁₃, and R₁₄ independently comprise hydrogen, aryl, C₁-C₆ alkyl, C₁-C₆ alkylaryl, C₁-C₆ alkoxy, or C₁-C₆ alkoxyaryl;
R₁₃ and R₁₄ may be taken together to form a ring having the formula —(CH₂)ₒ—X—(CH₂)ₚ-bonded to the nitrogen atom to which R₁₃ and R₁₄ are attached, and/or R₁₁ and R₁₂ may, independently, be taken together to form a ring having the formula —(CH₂)ₒ—X—(CH₂)ₚ— bonded to the atoms to which R₁₁ and R₁₂ are connected, wherein o and p are, independently, 1, 2, 3, or 4; X comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 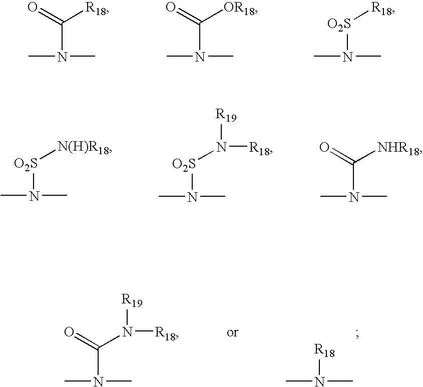
wherein the aryl and/or alkyl group(s) in R₁, R₂, R₃, R₅, R₆, R₇, R₈, R₉, R₁₀, R₁₁, R₁₂, R₁₃, R₁₄ R₁₅, R₁₆, R₁₇, R₁₈, and R₁₉ may be optionally substituted 1-4 times with a substituent group, wherein said substituent group(s) or the term substituted refers to groups comprising:
a) —H;
b) -Z-C₁₋₆ alkyl;
-Z-aryl;
-Z-C₁₋₆ alkylaryl;
-Z-C₁₋₆-alkyl-NR₂₀R₂₁;
-Z-C₁₋₆-alkyl-W—R₂₂;
wherein Z and W independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—, 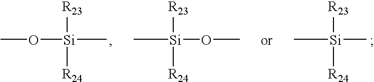
wherein;
R₂₀ and R₂₁ independently comprise hydrogen, aryl, C₁-C₆ alkyl, C₁-C₆ alkylaryl, C₁-C₆ alkoxy, or C₁-C₆ alkoxyaryl;
R₂₂, R₂₃, and R₂₄ independently comprise aryl, C₁-C₆ alkyl, C₁-C₆ alkylaryl, C₁-C₆ alkoxy, or C₁-C₆ alkoxyaryl; or
c) halogen, hydroxyl, cyano, carbamoyl, or carboxyl; and
R₂₀ and R₂₁ may be taken together to form a ring having the formula —(CH₂)_{q}—X—(CH₂)ᵣ-bonded to the nitrogen atom to which R₂₀ and R₂, are attached wherein q and r are, independently, 1, 2, 3, or 4; X comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 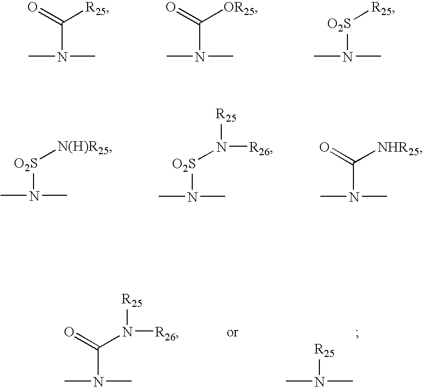
R₂₅, R₂₆, and R₂₇ independently comprise hydrogen, aryl, C₁-C₆ alkyl, or C₁-C₆ alkylaryl;
or a pharmaceutically acceptable salt, solvate or prodrug thereof.

16. The composition of claim 9, wherein the RAGE antagonist comprises compounds of Formula (III) 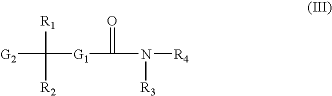
wherein for compound of Formula (III)
G₁ comprises C₁-C₆ alkylene or (CH₂)ₖ, where k is 0 to 3;
G₂ comprises a) hydrogen
b) —C₁₋₆ C1-6 alkyl;
c) -aryl;
d) —C₁₋₆ alkylaryl 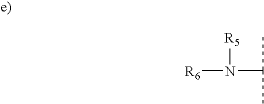
where R₅ and R₆ independently comprise
i) —H;
ii) —C₁₋₆ alkyl;
iii) -aryl;
iv) —C₁₋₆ alkylaryl;
v) —C(O)—O—C₁₋₆ alkyl;
vi) —C(O)—O—C₁₋₆ alkylaryl;
vii) —C(O)—O—C₁₋₆ alkylcycloalkylaryl;
viii) —C(O)—NH—C₁₋₆ alkyl;
ix) —C(O)—NH—C₁₋₆ alkylaryl;
x) —SO₂—C₁₋₆ alkyl;
xi) —SO₂—C₁₋₆ alkylaryl;
xii) —SO₂-aryl;
xiii) —SO₂—NH—C₁₋₆ alkyl;
xiv) —SO₂—NH—C₁₋₆ alkylaryl; 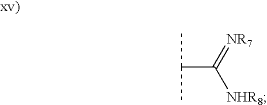
xvi) —C(O)—C₁₋₆ alkyl; or
xvii) —C(O)—C₁₋₆ alkylaryl; or
f) a group of the formula 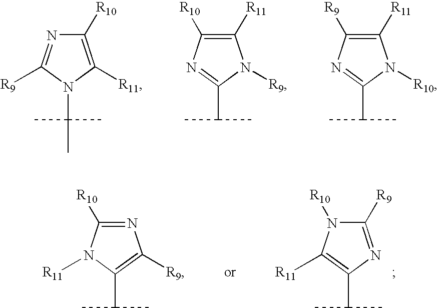
wherein
R₉, R₁₀, and R₁₁ may comprise hydrogen; or
R₉, R₁₀, and R₁₁ independently comprise
i) —C₁₋₆ alkyl;
ii) -aryl;
iii) —C₁₋₆ alkylaryl;
iv) —C(O)—O—C₁₋₆ alkyl;
v) —C(O)—O—C₁₋₆ alkylaryl;
vi) —C(O)—NH—C₁₋₆ alkyl;
vii) —C(O)—NH—C₁₋₆ alkylaryl;
viii) —SO₂—C₁₋₆ alkyl;
ix) —SO₂—C₁₋₆ alkylaryl;
x) —SO₂-aryl;
xi) —SO₂—NH—C₁₋₆ alkyl;
xii) —SO₂—NH—C₁₋₆ alkylaryl;
xiii) —C(O)—C₁₋₆ alkyl; or
xiv) —C(O)—C₁₋₆ alkylaryl;
or R₁₀ and R₁₁ may be taken together to constitute a fused cycloalkyl, fused heterocyclyl, or fused aryl ring containing the atoms to which R₁₀ and R₁₁ are bonded;
R₁ comprises
a) hydrogen;
b) —C₁₋₆ alkyl;
c) -aryl; or
d) —C₁₋₆ alkylaryl;
R₂ comprises
a) —C₁₋₆ alkyl;
b) -aryl;
c) —C₁₋₆ alkylaryl; or
d) a group of the formula 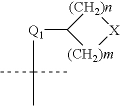
wherein m and n are independently selected from 1, 2, 3, or 4; X comprises a direct bond, CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 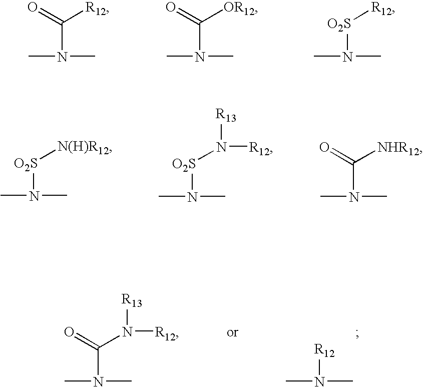
Q₁— comprises C₁₋₆ alkylene, C₂₋₆ alkenylene, or C₂₋₆ alkynylene;
R₃ comprises
a) hydrogen;
b) —C₁₋₆ alkyl;
c) —C₁₋₆ alkylaryl; or
d) —C₁₋₆ alkoxyaryl;
R₄ comprises
a) —C₁₋₆ alkylaryl;
b) —C₁₋₆ alkoxyaryl; or
c) -aryl;
R₇, R₈, R₁₂ and R₁₃ independently comprise hydrogen, C₁-C₆ alkyl, C₁-C₆ alkylaryl, or aryl; and wherein
the aryl and/or alkyl group(s) in R₁, R₂, R₃, R₄, R₅, R₆, R₇, R₈, and R₉, R₁₀, R₁₁, and R₁₂, and R₁₃ may be optionally substituted 1-4 times with a substituent group, wherein said substituent group(s) or the term substituted refers to groups comprising:
a) —H;
b) —Y—C₁₋₆ alkyl;
—Y-aryl;
—Y—C₁₋₆ alkylaryl;
—Y—C₁₋₆-alkyl-NR₁₄R₁₅;
—Y—C₁₋₆-alkyl-W—R₁₆;
wherein Y and W independently comprise —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—, 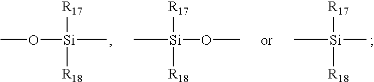
R₁₆, R₁₇, and R₁₈ comprise hydrogen, aryl, C₁-C₆ alkyl, C₁-C₆ alkylaryl, C₁-C₆ alkoxy, or C₁-C₆ alkoxyaryl; or
c) halogen, hydroxyl, cyano, carbamoyl, or carboxyl; and
R₁₄ and R₁₅ independently comprise hydrogen, aryl, C₁-C₆ alkyl, or C₁-C₆ alkylaryl; and wherein
R₁₄ and R₁₅ may be taken together to form a ring having the formula —(CH₂)ₒ-Z-(CH₂)ₚ-bonded to the nitrogen atom to which R₁₄ and R₁₅ are attached, and/or R₇ and R₈ may, independently, be taken together to form a ring having the formula —(CH₂)ₒ-Z-(CH₂)ₚ-bonded to the atoms to which R₇ and R₈ are attached, wherein o and p are, independently, 1, 2, 3, or 4; Z comprises a direct bond, —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 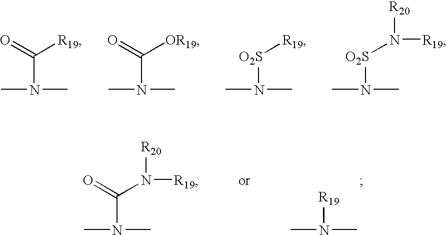
R₁₉ and R₂₀ independently comprise hydrogen, aryl, C₁-C₆ alkyl, or C₁-C₆ alkylaryl or a pharmaceutically acceptable salt, solvate or prodrug thereof.

17. The composition of claim 9, wherein the RAGE antagonist comprises compounds of Formula (IV) 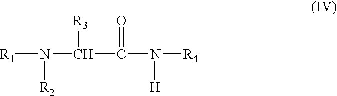
wherein,
R₁ and R₂ are independently selected from
a) —H;
b) —C₁₋₆ alkyl;
c) -aryl;
d) —C₁₋₆ alkylaryl;
e) —C(O)—O—C₁₋₆ alkyl;
f) —C(O)—O—C₁₋₆ alkylaryl;
g) —C(O)—NH—C₁₋₆ alkyl;
h) —C(O)—NH—C₁₋₆ alkylaryl;
i) —SO₂—C₁₋₆ alkyl;
j) —SO₂—C₁₋₆ alkylaryl;
k) —SO₂-aryl;
l) —SO₂—NH—C₁₋₆ alkyl;
m) —SO₂—NH—C₁₋₆ alkylaryl; 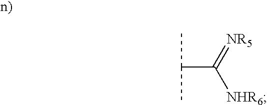
o) —C(O)—C₁₋₆ alkyl; and
p) —C(O)—C₁₋₆ alkylaryl;
R₃ is selected from
a) —C₁₋₆ alkyl;
b) -aryl; and
c) —C₁₋₆ alkylaryl;
R₄ is selected from
a) —C₁₋₆ alkylaryl;
b) —C₁₋₆ alkoxyaryl; and
c) -aryl;
R₅ and R₆ are independently selected from the group consisting of hydrogen, C₁-C₆ alkyl, C₁-C₆ alkylaryl, and aryl; and wherein
the aryl and/or alkyl group(s) in R₁, R₂, R₃, R₄, R₅, R₆, R₇, R₈, R₉, R₁₀, R₁₈, R₁₉, and R₂₀ may be optionally substituted 1-4 times with a substituent group, wherein said substituent group(s) or the term substituted refers to groups selected from the group consisting of:
a) —H;
b) —Y—C₁₋₆ alkyl;
—Y-aryl;
—Y—C₁₋₆ alkylaryl;
—Y—C₁₋₆-alkyl-NR₇R₈; and
—Y—C₁₋₆-alkyl-W—R₂₀;
wherein Y and W are, independently selected from the group consisting of —CH₂—, —O—, —N(H), —S—, SO₂—, —CON(H)—, NHC(O)—, —NHCON(H)—, —NHSO₂—, —SO₂N(H)—, —C(O)—O—, NHSO₂NH—, —O—CO—, 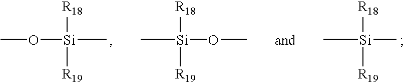
and
c) halogen, hydroxyl, cyano, carbamoyl, or carboxyl; and
R₁₈ and R₁₉ are independently selected from the group consisting of aryl, C₁-C₆ alkyl, C₁-C₆ alkylaryl, C₁-C₆ alkoxy, and C₁-C₆ alkoxyaryl;
R₂₀ is selected from the group consisting of aryl, C₁-C₆ alkyl, and C₁-C₆ alkylaryl;
R₇, R₈, R₉ and R₁₀ are independently selected from the group consisting of hydrogen, aryl, C₁-C₆ alkyl, and C₁-C₆ alkylaryl; and wherein
R₇ and R₈ may be taken together to form a ring having the formula —(CH₂)ₘ—X—(CH₂)ₙ— bonded to the nitrogen atom to which R₇ and R₈ are attached, and/or R₅ and R₆ may, independently, be taken together to form a ring having the formula —(CH₂)ₘ—X—(CH₂)ₙ— bonded to the nitrogen atoms to which R₅ and R₆ are attached, wherein m and n are, independently, 1, 2, 3, or 4; X is selected from the group consisting of —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 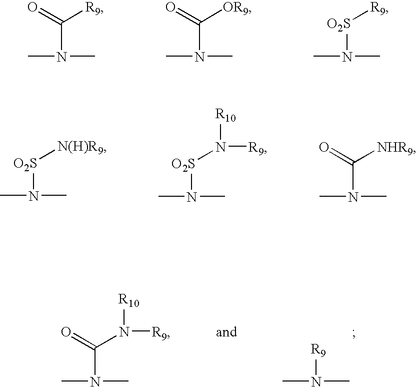
or a pharmaceutically acceptable salt, solvate or prodrug thereof.

18. The composition of claim 1, wherein the RAGE antagonist comprises a polypeptide or peptidomimetic.

19. The composition of claim 18, wherein the polypeptide or peptidomimetic comprises sRAGE or a fragment thereof.

20. The composition of claim 18, wherein the polypeptide or peptidomimetic comprises the V-domain of sRAGE.

21. The composition of claim 18, wherein the polypeptide or peptidomimetic comprises an anti-RAGE antibody, or a fragment thereof.

22. The composition of claim 19, wherein the sRAGE or a fragment thereof is linked to fragment of immunoglobulin.

23. The composition of claim 1, wherein the RAGE antagonist is administered as a dose ranging from 0.01 to 500 mg/kg per day.

24. The composition of claim 1, wherein the RAGE antagonist is administered as a dose ranging from 0.1 to 200 mg/kg per day.

25. The composition of claim 1, wherein the RAGE antagonist is administered as a dose ranging from 1 to 100 mg/kg per day.

26. The composition of claim 1, wherein the RAGE antagonist is administered as a dose ranging from about 5 to about 20 mg/kg per day.

27. The composition of claim 1, wherein the composition is suitable for administration by a topical route.

28. The composition of claim 1, wherein the composition is suitable for administration by an intravenous route.

29. The composition of claim 1, wherein the composition is suitable for oral administration.

30. The composition of claim 1, wherein the composition is suitable for transdermal administration.

31. The composition of claim 1, wherein the composition is suitable for subcutaneous administration.

32. The composition of claim 1, further comprising a second therapeutic agent.

33. The composition of claim 32, wherein the second therapeutic agent comprises a compound effective in treating Aβ amyloidosis.

34. The composition of claim 33, wherein the second therapeutic agent comprises a cholinesterase inhibitor, an antipsychotic, an antidepressant, or an anticonvulsant.

35. The composition of claim 32, wherein the second therapeutic agent comprises a compound effective in treating amyloid-light chain (AL) amyloidosis.

36. The composition of claim 35, wherein the second therapeutic agent comprises an alkylating agent, an antibiotic, an antimetabolite, a plant alkaloid, a hormone, or a biologic response modifier such as an interferon or an interleukin.

37. The composition of claim 32, wherein the second therapeutic agent comprises a compound effective in treating amyloid-associated (AA) amyloidosis.

38. The composition of claim 37, wherein the second therapeutic agent comprises an analgesic, a nonsteroidal anti-inflammatory drug (NSAID), a disease-modifying antirheumatic drug (DMARD), or a biologic response modifier.

39. A composition to inhibit the onset and/or progression of amyloidosis in an individual comprising a pharmacologically effective amount of a RAGE antagonist in a pharmaceutically acceptable carrier, wherein a pharmacologically effective amount of antagonist comprises sufficient RAGE antagonist to reduce amyloid plaque formation in the individual.

40. The composition of claim 39, wherein the RAGE antagonist inhibits symptoms associated with amyloidosis.

41. A method to reverse amyloidosis in an individual in need thereof comprising administering a pharmacologically effective amount of a RAGE antagonist in a pharmaceutically acceptable carrier to the individual, wherein a pharmacologically effective amount of RAGE antagonist reduces pre-existing amyloid plaques in the individual.

42. A method to inhibit the onset and/or progression of amyloidosis in an individual comprising administering a pharmacologically effective amount of a RAGE antagonist in a pharmaceutically acceptable carrier to the individual, wherein a pharmacologically effective amount of RAGE antagonist comprises sufficient RAGE antagonist to reduce amyloid plaque formation in the individual.
